(19) United States
(12) Patent Application Publication
(10) Pub. No.: US 2025/0345009 A1
(43) Pub. Date: Nov. 13, 2025

(54) CONVERSATIONAL CLINICAL TRIAL MANAGEMENT SYSTEM AND METHOD

(71) Applicant: UpDoc Inc., Los Altos, CA (US)

(72) Inventors: Sharif Vakili, Los Altos, CA (US); Ashwin K. Nayak, Mountain View, CA (US)

(21) Appl. No.: 19/273,011

(22) Filed: Jul. 17, 2025

Related U.S. Application Data
(63) Continuation-in-part of application No. PCT/US24/53096, filed on Oct. 25, 2024.
Application No. PCT/US24/53096 is a continuation of application No. 18/384,321, filed on Oct. 26, 2023, now Pat. No. 12,251,242.
(60) Provisional application No. 63/546,905, filed on Nov. 1, 2023.
(60) Provisional application No. 63/609,269, filed on Dec. 12, 2023.

(51) Int. Cl. A61B 5/00; A61B 5/145; G16H 40/67

(52) U.S. Cl. CPC A61B 5/7465; A61B 5/0004; A61B 5/0022; A61B 5/14532; A61B 5/741; A61B 5/749; G16H 40/67

(57) ABSTRACT

A conversational AI platform for managing remote and hybrid clinical trials for investigational medical interventions. Embodiments of the present disclosure comprise a conversational AI model and an algorithmic logic engine configured to define and implement protocol parameters for dosing schemas, visit schedules, safety thresholds, and eligibility criteria for a clinical trial. An AI agent is configured to deliver mapped voice prompts to participant devices, captures audio responses, transcribe and extract symptom, adherence, or adverse-event data, and pair response data with physiological-sensor or laboratory input data. The logic engine continuously evaluates the combined data to adaptively select dose-escalation or titration instructions and issue follow-up queries while enforcing safety thresholds. All prompts, audio, transcriptions, decisions, and metadata may be immutably timestamped in an electronic record repository accessible via role-based, encrypted connections. Embodiments of the present disclosure provide for real-time, audit-ready trial communications, automated personalized dosing, and enhanced participant safety monitoring.

### CROSS-REFERENCE TO RELATED APPLICATIONS

This application is a continuation-in-part of PCT Application Number PCT/US24/53096, filed on Oct. 25, 2024, entitled “ARTIFICIALLY INTELLIGENT SYSTEM FOR MEDICATION MANAGEMENT”; said application claiming priority benefit of U.S. application Ser. No. 18/384,321 filed Oct. 26, 2023; U.S. Provisional App. Ser. No. 63/546,905, filed Nov. 1, 2023; and U.S. Provisional App. Ser. No. 63/609,269 filed Dec. 12, 2023; the entireties of which applications are hereby incorporated herein at least by virtue of this reference.

### FIELD

The present disclosure relates to the field of computerized methods and systems for clinical trial management; in particular, an artificially-intelligent, conversational system and method for automated remote management of clinical trials.

### BACKGROUND

Clinical trials are the primary vehicle by which investigational drugs, biologics, and combination products demonstrate safety and efficacy before marketing approval. Early-phase trials (e.g., Phase I, first-in-human studies) typically employ dose-escalation designs to identify a maximum tolerated dose or a biologically effective dose, while later-phase studies often incorporate dose-titration rules to individualize therapy and optimize the risk/benefit profile across heterogeneous participants. Conventional approaches rely on static, protocol-defined dose grids and manual review of adverse-event logs or pharmacokinetic (PK) snapshots. Investigators frequently enter dosing decisions into disparate spreadsheets or electronic data-capture (EDC) systems, then transmit instructions to pharmacy or nursing staff through e-mail or handwritten orders. These disconnected workflows are prone to transcription errors, version-control problems, and latency that can delay next-dose administration.

Clinical trials sponsors have attempted to digitize dosing workflows by layering simple rule engines onto existing EDC platforms or by deploying standalone “dose calculator” applications. While such tools can automate basic arithmetic (e.g., body-surface-area scaling), they seldom integrate real-time physiologic signals (such as heart-rate variability from wearables) or continuous laboratory feeds (such as near-patient PK assays). As a result, clinicians still perform ad-hoc data aggregation and qualitative risk assessment before approving an escalated dose. The cognitive burden grows rapidly when Bayesian adaptive designs, continual-reassessment methods, or model-informed drug development (MIDD) paradigms are adopted, because posterior probability updates and predictive exposure modelling must occur on a per-participant or per-cohort basis.

Compounding these operational challenges are stringent regulatory requirements for electronic records and electronic signatures. Under 21 CFR Part 11, all computer systems that create, modify, maintain, or transmit clinical trial data must generate secure, computer-readable audit trails; enforce robust, role-based access controls; and preserve records in a manner that is trustworthy, reliable, and readily retrievable throughout the retention period. Legacy dose-management spreadsheets and many bespoke dosing apps were never validated to this standard, forcing study teams either to print and wet-sign dosing worksheets or to undertake costly, post-hoc system validation and remediation.

Emerging trends further stress traditional infrastructures. Decentralized and hybrid trials distribute dosing activities across investigational sites, mobile research units, and even participants' homes. Combination products and cell- and gene-therapy regimens often require weight-based or biomarker-triggered dose adjustments spanning multiple infusion cycles. Risk-based monitoring guidance from the Food and Drug Administration (FDA) and the International Council for Harmonisation of Technical Requirements for Pharmaceuticals for Human Use (ICH) encourages near-real-time detection of protocol deviations or dosing-limit excursions, yet site staff frequently lack integrated dashboards that surface actionable insights. Finally, increasing public and regulatory scrutiny on data integrity amplifies the need for systems that capture and lock dosing decisions contemporaneously, with clear attribution to individual investigators and links to supporting source data.

### SUMMARY

The following presents a simplified summary of some embodiments of the invention in order to provide a basic understanding of the invention. This summary is not an extensive overview of the invention. It is not intended to identify key/critical elements of the invention or to delineate the scope of the invention. Its sole purpose is to present some embodiments of the invention in a simplified form as a prelude to the more detailed description that is presented later.

Certain aspects of the present disclosure provide for a computer-implemented method for clinical trial management. In accordance with certain aspects of the present disclosure, the computer-implemented method may comprise one or more steps or operations for receiving (e.g., by at least one processor via a network interface) protocol parameters that define one or more of a dosing schema, a visit schedule, one or more safety or escalation thresholds, and participant-eligibility criteria for a clinical trial for an investigational medical intervention. The computer-implemented method may further comprise one or more steps or operations for configuring (e.g., by the at least one processor) a conversational artificial intelligence (AI) model in accordance with the protocol parameters. In certain embodiments, configuring the conversational AI model comprises configuring a set of generative voice prompts mapped to the protocol parameters. The computer-implemented method may further comprise one or more steps or operations for configuring (e.g., by the at least one processor) an algorithmic logic engine in accordance with the protocol parameters. In certain embodiments, the algorithmic logic engine comprises one or more decision rules for processing participant reported data according to the protocol parameters. The computer-implemented method may further comprise one or more steps or operations for transmitting (e.g., to a participant device comprising a microphone and a speaker) a first generative voice prompt via a conversational AI agent executing the conversational AI model. The computer-implemented method may further comprise one or more steps or operations for receiving (e.g., via the participant device) audio data in response to the first generative voice prompt. The computer-implemented method may further comprise one or more steps or operations for transcribing the audio data into structured textual content and extracting participant data comprising at least one of symptom information, adherence confirmation, or adverse event descriptors. The computer-implemented method may further comprise one or more steps or operations for evaluating (e.g., by the algorithmic logic engine) the participant data in combination with objective clinical data received from a physiological sensor device or an external laboratory data feed against the protocol parameters. The computer-implemented method may further comprise one or more steps or operations for dynamically generating (e.g., via the conversational AI agent) a second generative voice prompt in response to evaluating the participant data. In certain embodiments, the second generative voice prompt comprises a dosage instruction for the investigational medical intervention, or a follow-up generative voice prompt configured to request additional information from the participant. The computer-implemented method may further comprise one or more steps or operations for recording (e.g., in an electronic record repository) (i) the protocol parameters, (ii) each generative voice prompt, (iii) the audio data, (iv) the structured textual content, (v) a timestamp, (vi) an audit trail indicator, and (vii) any dosage instruction. The computer-implemented method may further comprise one or more steps or operations for rendering the secure electronic record repository accessible to at least one authenticated sponsor device or investigator client device via a role-based network connection.

In accordance with certain embodiments of the present disclosure, the computer-implemented method may be further configured wherein the dosage instruction is dynamically selected according to an adaptive dose escalation model. In certain embodiments, the adaptive dose escalation model may comprise a Bayesian continual reassessment algorithm or a modified toxicity probability interval algorithm. In certain embodiments, the step of evaluating the participant data may further comprise one or more steps or operations for classifying adverse event descriptors by automatically mapping each descriptor to a severity grade. The computer-implemented method may further comprise one or more steps or operations for communicating an alert to the at least one authenticated sponsor device or the investigator client device when the severity grade meets or exceeds a prespecified threshold. In certain embodiments, the step of transcribing the audio data may further comprise one or more steps or operations for analyzing at least one vocal biomarker from the audio data to derive at least one psychophysiological indicator for the participant. The computer-implemented method may further comprise one or more steps or operations for generating (e.g., with the conversational AI agent) a follow-up generative voice prompt in response to the psychophysiological indicator exceeding a configurable threshold. In certain embodiments, the objective clinical data includes real-time physiological measurements received from the physiological sensor device at the participant device via a short-range wireless protocol, the real-time physiological measurements comprising at least one of heart rate, heart rate variability, physical activity level, sleep duration, or interstitial glucose. The computer-implemented method may further comprise one or more steps or operations for processing (e.g., by the at least one processor) the audio data to generate an encrypted audio file comprising the audio data and an encrypted text file comprising the structured textual content. The computer-implemented method may further comprise one or more steps or operations for configuring the dosage instruction according to the participant data and the protocol parameters. In certain embodiments, the dosage instruction may be configured by: (a) comparing current participant safety and efficacy markers to the one or more safety or escalation thresholds; (b) determining an individualized titration increment that does not exceed a protocol defined maximum dose; and (c) adjusting the individualized titration increment in response to each subsequently received set of participant data.

Further aspects of the present disclosure provide for a system for managing a clinical trial of an investigational medical intervention comprising at least one server comprising a processor, a non-transitory computer-readable medium, and a network interface, the non-transitory computer-readable medium comprising processor-executable instructions stored thereon that, when executed by the processor, cause the server to perform one or more operations. In accordance with certain embodiments, the one or more operations may comprise operations for receiving protocol parameters that define one or more of a dosing schema, a visit schedule, one or more safety or escalation thresholds, and participant eligibility criteria. The one or more operations may further comprise operations for configuring a conversational artificial intelligence (AI) model and an algorithmic logic engine in accordance with the protocol parameters. In certain embodiments, the operations for configuring the conversational AI model and the algorithmic logic engine may further comprise: (i) configuring a set of generative voice prompts mapped to protocol activities for execution by a conversational AI agent, and (ii) encoding decision rules that relate participant reported and objective clinical data to the dosing schema and the safety or escalation thresholds. The one or more operations may further comprise operations for storing (e.g., in an electronic record repository) the protocol parameters, the generative voice prompts, and the decision rules in a traceable format. In certain embodiments, the system may further comprise at least one participant device comprising a microphone, a speaker, and programmed circuitry. The programmed circuitry may be configured to receive from the server a first generative voice prompt; capture audio data from a participant in response to the first generative voice prompt; and transmit the captured audio data to the server via a secure network connection. In certain embodiments, the system may further comprise an audio-processing module executable by the server and configured to transcribe the audio data into structured textual content; and extract participant data comprising at least one of symptom information, adherence confirmation, or adverse event descriptors. In certain embodiments, the algorithmic logic engine is executable by the server and configured to evaluate the participant data in combination with objective clinical data received from at least one physiological sensor device or external laboratory feed against the safety or escalation thresholds and generate at least one of a dosage or scheduling instruction for the investigational medical intervention or a follow-up generative voice prompt. In certain embodiments, the system may further comprise a communication module executable by the server and configured to transmit the generated dosage or scheduling instruction or the follow-up generative voice prompt to the participant device. In certain embodiments, the system may further comprise an audit trail module executable by the server and configured to timestamp and immutably record in the electronic record repository (i) each generative voice prompt, (ii) each audio capture, (iii) the structured textual content, (iv) each generated dosage or scheduling instruction, and (v) metadata identifying the participant device session. In certain embodiments, the system may further comprise at least one sponsor or investigator client device configured to access, via a role-based, encrypted network connection, the electronic record repository and the audit trail data for review, monitoring, or regulatory inspection.

In accordance with certain aspects of the present disclosure, the system may be further configured wherein the dosage instruction is dynamically selected according to an adaptive dose escalation model. In certain embodiments, the adaptive dose escalation model comprises a Bayesian continual reassessment algorithm or a modified toxicity probability interval algorithm. In certain embodiments, the objective clinical data includes real-time physiological measurements received from the physiological sensor device at the participant device via a short-range wireless protocol, the real-time physiological measurements comprising at least one of heart rate, heart rate variability, physical activity level, sleep duration, or interstitial glucose. In certain embodiments, the audio-processing module is further configured to perform sentiment analysis and vocal biomarker extraction on the received audio data to derive at least one psychophysiological indicator selected from the group consisting of stress level, fatigue, and depressive affect. In certain embodiments, the algorithmic logic engine is further configured to trigger a follow-up generative voice prompt when the psychophysiological indicator surpasses a configurable threshold. In certain embodiments, the algorithmic logic engine may be further configured to enforce a cumulative dose ceiling by: (i) maintaining, in the electronic record repository, a running total of the participant's administered dose of the investigational medical intervention over a specified interval; and (ii) automatically suspending further upward titration and generating an electronic alert to an investigator client device when the cumulative total reaches or exceeds a predefined maximum allowable exposure. In certain embodiments, the algorithmic logic engine may be further configured to (i) downgrade a dose level for the investigational medical intervention in response to detecting a dose-limiting toxicity event based on the participant data and the objective clinical data, (ii) configure a lockout action for subsequent dose escalation, and (iii) record the lockout action, the dose-limiting toxicity event data, and timestamp in an audit trail portion of the electronic record repository.

Still further aspects of the present disclosure provide for a non-transitory computer readable medium comprising processor-executable instructions stored thereon that, when executed by at least one processor, are configured to cause the at least one processor to perform one or more operations of a computer-implemented method for clinical trial management. In accordance with certain embodiments, the one or more operations may comprise operations for receiving protocol parameters that define one or more of a dosing schema, a visit schedule, one or more safety or escalation thresholds, and participant eligibility criteria for a clinical trial for an investigational medical intervention. The one or more operations may comprise operations for configuring a conversational artificial intelligence (AI) model in accordance with the protocol parameters. In certain embodiments, configuring the conversational AI model may comprise configuring a set of generative voice prompts mapped to the protocol parameters. The one or more operations may comprise operations for configuring an algorithmic logic engine in accordance with the protocol parameters. In certain embodiments, the algorithmic logic engine comprises one or more decision rules for processing participant reported data according to the protocol parameters. The one or more operations may comprise operations for transmitting (e.g., to a participant device comprising a microphone and a speaker) a first generative voice prompt via a conversational AI agent executing the conversational AI model. The one or more operations may comprise operations for receiving (e.g., via the participant device) audio data in response to the first generative voice prompt. The one or more operations may comprise operations for transcribing the audio data into structured textual content and extracting participant data comprising at least one of symptom information, adherence confirmation, or adverse event descriptors. The one or more operations may comprise operations for evaluating (e.g., by the algorithmic logic engine) the participant data in combination with objective clinical data received from a physiological sensor device or an external laboratory data feed against the protocol parameters. The one or more operations may comprise operations for dynamically generating a second generative voice prompt in response to evaluating the participant data. In certain embodiments, the second generative voice prompt comprises a dosage instruction for the investigational medical intervention, or a follow-up generative voice prompt configured to request additional information from the participant. The one or more operations may comprise operations for recording (e.g., in an electronic record repository) (i) the protocol parameters, (ii) each generative voice prompt, (iii) the audio data, (iv) the structured textual content, (v) a timestamp, (vi) an audit trail indicator, and (vii) any dosage instruction. The one or more operations may comprise operations for rendering the secure electronic record repository accessible to at least one authenticated sponsor device or investigator client device via a role-based network connection.

The foregoing has outlined rather broadly the more pertinent and important features of the present invention so that the detailed description of the invention that follows may be better understood and so that the present contribution to the art can be more fully appreciated. Additional features of the invention will be described hereinafter which form the subject of the claims of the invention. It should be appreciated by those skilled in the art that the conception and the disclosed specific methods and structures may be readily utilized as a basis for modifying or designing other structures for carrying out the same purposes of the present invention. It should be realized by those skilled in the art that such equivalent structures do not depart from the spirit and scope of the invention as set forth in the appended claims.

### BRIEF DESCRIPTION OF DRAWINGS

The skilled artisan will understand that the figures, described herein, are for illustration purposes only. It is to be understood that in some instances various aspects of the described implementations may be shown exaggerated or enlarged to facilitate an understanding of the described implementations. In the drawings, like reference characters generally refer to like features, functionally similar and/or structurally similar elements throughout the various drawings. The drawings are not necessarily to scale, emphasis instead being placed upon illustrating the principles of the teachings. The drawings are not intended to limit the scope of the present teachings in any way. The system and method of the present disclosure may be better understood from the following illustrative description with reference to the following drawings in which:

FIG. 1 is an architecture diagram of a conversational system for automated remote management of clinical trials;

FIG. 2 is a functional block diagram of a conversational system for automated remote management of clinical trials, in accordance with certain aspects of the present disclosure;

FIG. 3 is a process flow diagram of a conversational system and method for automated remote management of clinical trials, in accordance with certain aspects of the present disclosure;

FIG. 4A is a logic flow diagram of a routine of the system for automated remote management of clinical trials;

FIG. 4B is a logic flow diagram of a branch routine of the system for automated remote management of clinical trials;

FIG. 5 is a process flow diagram of an audio processing routine for the conversational system for automated remote management of clinical trials;

FIG. 6 is a logic flow diagram of a routine of the system for automated remote management of clinical trials;

FIG. 7 is a logic flow diagram of a routine of the system for automated remote management of clinical trials;

FIG. 8 is a logic flow diagram of a routine of the system for automated remote management of clinical trials;

FIG. 9 is a logic flow diagram of a routine of the system for automated remote management of clinical trials;

FIG. 10 is a block diagram illustrating a representative software architecture, which may be used in conjunction with various hardware architectures herein described; and

FIG. 11 is a block diagram illustrating components of a machine, according to some exemplary embodiments, able to read instructions from a machine-readable medium (e.g., a machine-readable storage medium) and perform any one or more of the methodologies discussed herein.

### DETAILED DESCRIPTION

It should be appreciated that all combinations of the concepts discussed in greater detail below (provided such concepts are not mutually inconsistent) are contemplated as being part of the inventive subject matter disclosed herein. It also should be appreciated that terminology explicitly employed herein that also may appear in any disclosure incorporated by reference should be accorded a meaning most consistent with the particular concepts disclosed herein.

Following below are more detailed descriptions of various concepts related to, and embodiments of, inventive methods, apparatus and systems configured to provide for a conversational AI platform for managing remote and hybrid clinical trials for investigational medical interventions. Embodiments of the present disclosure comprise a conversational AI model and an algorithmic logic engine configured to define and implement protocol parameters for dosing schemas, visit schedules, safety thresholds, and eligibility criteria for a clinical trial. An AI agent is configured to deliver mapped voice prompts to participant devices, captures audio responses, transcribe and extract symptom, adherence, or adverse-event data, and pair response data with physiological-sensor or laboratory input data. The logic engine continuously evaluates the combined data to adaptively select dose-escalation or titration instructions and issue follow-up queries while enforcing safety thresholds. All prompts, audio, transcriptions, decisions, and metadata may be immutably timestamped in an electronic record repository accessible via role-based, encrypted connections. Embodiments of the present disclosure provide for real-time, audit-ready trial communications, automated personalized dosing, and enhanced participant safety monitoring.

It should be appreciated that various concepts introduced above and discussed in greater detail below may be implemented in any of numerous ways, as the disclosed concepts are not limited to any particular manner of implementation. Examples of specific implementations and applications are provided primarily for illustrative purposes. The present disclosure should in no way be limited to the exemplary implementation and techniques illustrated in the drawings and described below.

Before the present invention and specific exemplary embodiments of the invention are described, it is to be understood that this invention is not limited to the particular embodiments described, and as such may vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range is encompassed by the invention. The upper and lower limits of these smaller ranges may independently be included in the smaller ranges, and are also encompassed by the invention, subject to any specifically excluded limit in a stated range. Where a stated range includes one or both of the endpoint limits, ranges excluding either or both of those included endpoints are also included in the scope of the invention.

As used herein, “exemplary” means serving as an example or illustration and does not necessarily denote ideal or best.

As used herein, the term “includes” means includes but is not limited to, the term “including” means including but not limited to. The term “based on” means based at least in part on.

As used herein, the term “interface” refers to any shared boundary across which two or more separate components of a computer system may exchange information. The exchange can be between software, computer hardware, peripheral devices, humans, and combinations thereof.

As used herein, the term “protocol parameter(s)” refers to any set of data values, such as dosing schema, visit schedule, safety or escalation thresholds, and eligibility criteria, that operationally configure one or more workflow executed by embodiments of the present disclosure.

As used herein, the term “dosing schema” refers to a predefined arrangement of dose amounts and timing for administration of an investigational medical intervention.

As used herein, the term “safety or escalation threshold” refers to a quantitative or qualitative limit that, when reached, causes a dose-escalation, de-escalation, titration, or lockout action according to protocol rules.

As used herein, the term “conversational artificial intelligence model” or “conversational AI model” or “AI model” refers to a trained machine-learning model capable of generating and understanding natural-language voice prompts for real-time dialogue with a participant.

As used herein, the term “generative voice prompt” refers to an audio prompt whose linguistic content is produced or selected on-the-fly by the conversational AI model in response to current trial data.

As used herein, the term “algorithmic logic engine” refers to executable software logic that applies protocol-encoded decision rules to participant and clinical data to generate dosing or follow-up actions.

As used herein, the term “participant data” refers to data that originates from or characterizes a participant and includes at least symptom information, adherence confirmation, adverse-event descriptors, and optionally psychophysiological indicators.

As used herein, the term “objective clinical data” refers to numerical or categorical measurements obtained from a physiological sensor device or external laboratory source, exclusive of participant self-reports.

As used herein, the terms “adaptive dose escalation model” or “Bayesian continual reassessment algorithm” or “modified toxicity probability interval algorithm” refer to statistical methods that update dose-level recommendations using accumulating safety or efficacy data to approach a target toxicity or response probability.

As used herein, the terms “psychophysiological indicator” or “vocal biomarker” refer to a quantitative feature extracted from audio (e.g., pitch variability, speech rate) that correlates with a participant's physiological or psychological state.”

As used herein, the terms “participant device” or “sponsor device” or “investigator client device” refer to a computing device, respectively operated by a participant, sponsor, or investigator, that communicates with the server via a secure network connection.

As used herein, the terms “electronic record repository” or “audit-trail module” refer to a tamper-evident data store that persistently logs all trial-related data items together with cryptographically bound timestamps and change history.

As used herein, the term “short-range wireless protocol” refers to a radio communication protocol having an effective range under 100 meters, such as BLUETOOTH Low Energy.

As used herein, the term “investigational medical intervention” refers to a drug, biologic, device, procedure, or combination thereof that has not yet received full regulatory marketing approval and is administered or applied under a controlled clinical protocol to evaluate its safety, efficacy, dosage, or performance characteristics.

Certain exemplary embodiments according to the principles herein may include computerized methods, systems and non-transitory computer-readable media that automate remote conduct of a clinical trial for an investigational medical intervention. In one aspect, a server receives protocol parameters, such as dosing schema, visit schedule, escalation or safety thresholds, and eligibility criteria, and uses them to configure (i) a conversational artificial-intelligence (AI) model that maps protocol activities to generative voice prompts and (ii) an algorithmic logic engine that encodes decision rules tied to those parameters. During the trial, the server may transmit a first voice prompt to a participant device, capture spoken responses, transcribe the audio into structured text, extracts symptom, adherence or adverse-event data, and merge that data with objective clinical inputs from connected physiological sensors and/or laboratory feeds. The algorithmic logic engine may evaluate a combined dataset against the protocol parameters and dynamically issue a dosage or scheduling instruction or a follow-up prompt, thereby enabling individualized dose-escalation or titration in real time. Exemplary escalation models may include Bayesian continual-reassessment and modified toxicity-probability-interval algorithms. Adverse events may be automatically classified by severity, and alerts may be pushed to sponsor or investigator dashboards when user-defined thresholds are exceeded. The algorithmic logic engine may also enforce cumulative-dose ceilings and lockouts on further escalation upon detecting a dose-limiting toxicity event. In accordance with certain embodiments, all protocol parameters, prompts, audio captures, transcriptions, generated instructions and metadata may be immutably time-stamped and written to an electronic record repository with an integrated audit-trail module. Authorized sponsor and investigator clients gain role-based, encrypted access for monitoring or regulatory inspection.

Certain benefits and advantages of the present disclosure include methods and systems for automating remote participant engagement through conversational AI, thereby reducing site burden and increasing adherence of participants in remote clinical trials.

Certain benefits and advantages of the present disclosure include methods and systems for delivering real-time, adaptive dose-escalation or titration (e.g., in dose-escalation design trials) that optimizes therapeutic exposure while minimizing participant risk.

Certain benefits and advantages of the present disclosure include methods and systems for integrating multimodal sensor and laboratory data for continuous safety surveillance and early adverse-event detection.

Certain benefits and advantages of the present disclosure include methods and systems for generating an immutable, audit-ready electronic record that streamlines regulatory inspections and demonstrates Good Clinical Practice compliance.

Certain benefits and advantages of the present disclosure include methods and systems for providing role-based, encrypted access for sponsors and investigators to enhance transparency and data-driven decision making across geographically dispersed clinical trial teams.

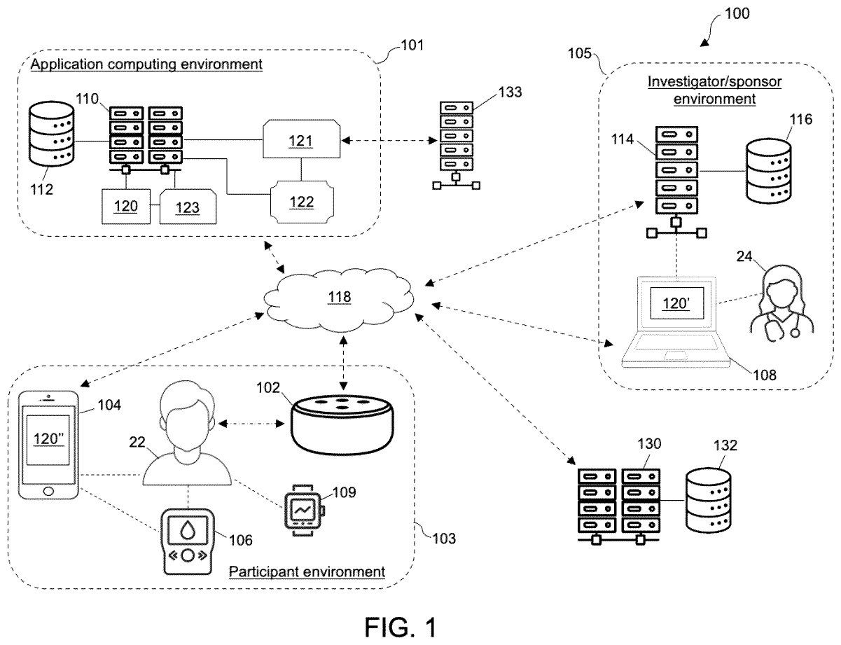

Turning now descriptively to the drawings, in which similar reference characters denote similar elements throughout the several views, FIG. 1 depicts an architecture diagram of a conversational system 100 for automated remote management of clinical trials. In accordance with certain aspects of the present disclosure, system 100 is configured to enable a conversational (i.e., voice-based or text-based) system for remote management of a clinical trial for an investigation medical intervention. System 100 may comprise an investigator/sponsor computing environment 105, a participant computing environment 103 and an application computing environment 101 configured to enable configuration and implementation of a clinical trial protocol for investigation medical intervention (e.g., by an investigator user 24). In accordance with certain aspects of the present disclosure, the clinical trial protocol may comprise one or more functional areas including, but not limited to, (i) participant consent and onboarding, (ii) remote eligibility screening (i.e., recruitment), (iii) dosing instructions and reminders, (iv) collection of participant-reported outcomes and medication logs, (v) safety monitoring and adverse event triage, (vi) adaptive dose-escalation (e.g., within adaptive-design trials), (vii) clinical check-in protocols (e.g., virtual office visits), (viii) sample collection and/or lab testing protocols, (ix) participant engagement, retention and/or adherence protocols, (x) translation and transcription for multilingual global trials, (xi) protocol-deviation and reflex logic, (xii) data treatment protocols for electronic data capture (EDC) and clinical trial management systems (CTMS), (xiii) post-market surveillance protocols, and (xiv) real-world evidence generation protocols.

In accordance with certain aspects of the present disclosure, user 24 may comprise one or more stakeholders within a clinical trial ecosystem including, but not limited to, a sponsor user, a principal investigator (PI) user, a study team user, site staff users, institutional review board (IRB) user, a regulatory agency user, a clinical research organization (CRO) user, a data safety monitoring board (DSMB) user, a pharmacovigilance team user, a statistician user, a clinical practitioner user, a laboratory user, a data management user, a patient advocacy group user, and/or a payor user. In accordance with certain embodiments, participant environment 103 may comprise a smart speaker 102, a participant client 104, a testing device 106 (e.g., a continuous glucose monitor) and a sensor device 109 (e.g., a wearable activity tracker). Participant client 104 may comprise a smart phone, tablet computer, desktop computer, personal digital assistant, or other personal computing device. In certain embodiments, testing device 106 and/or sensor device 109 may be communicably engaged with participant client 104 via a wired or wireless data transfer interface (e.g., BLUETOOTH low energy) to transmit physiological, biological and/or activity sensor data for participant user 22 to participant client 104.

In accordance with certain embodiments, an investigator environment 105 may include an investigator computing device 108, a clinical trial management server 114 and a clinical trial management database 116. Investigator computing device 108 may be communicably engaged with clinical trial management server 114 via a local area or a wide area network interface. Clinical trial management database 116 may be communicably engaged with clinical trial management server 114 to store and retrieve clinical trial management data in association with a clinical trial for an investigational medical intervention. Investigator computing device 108, clinical trial management server 114 and clinical trial management database 116 may be operably engaged according to a HIPAA-compliant network architecture. In accordance with certain aspects of the present disclosure, clinical trial management server 114 and clinical trial management database 116 may comprise a plurality of system configurations and protocols to ensure compliance with 21 CFR Part 11. In certain embodiments, system 100 may comprise one or more external electronic medical record (EMR)/electronic health record (EHR) server 130 and external EMR/EHR database 132. External EMR/EHR server 130 and external EMR/EHR database 132 may comprise one or more third-party medical server, including one or more laboratory information management system (LIMS) server, third-party payor server, regulatory agency server, and the like.

In accordance with certain aspects of the present disclosure, participant environment 103, investigator/sponsor environment 105 and, optionally, external EMR/EHR server 130, may be communicably engaged with application computing environment 101 via a communications network 118. Application computing environment 101 may comprise a cloud computing environment. Communications network 118 may comprise one or more network interfaces to enable one or more real-time data transfer interfaces between participant environment 103, investigator/sponsor environment 105 and external EMR/EHR server 130 including, for example, one or more application programming interface (API) or software development kit (SDK). In accordance with certain aspects of the present disclosure, application computing environment 101 comprises at least one application server 110 and an application database 112. In accordance with certain embodiments, application database 112 may comprise a knowledge base comprising a plurality of subject-matter information from which a conversational AI model may draw to generate responses to one or more user queries. Application server 110 may comprise one or more computing modules and control services to enable one or more functions and operations of system 100. In accordance with certain aspects of the present disclosure, application server 110 comprises a clinical trial management application 120, an algorithmic logic engine 123, a conversational AI engine 121, and a conversational AI agent 122 service. In certain embodiments, conversational AI engine 121 may comprise an ensemble of large language models configured to drive a plurality of generative text-to-speech, text-to-text and/or speech-to-speech outputs of conversational AI agent 122. In accordance with certain aspects of the present disclosure, system 100 may comprise an external server 133 comprising one or more third-party service; for example, a large language model service, a know-your-customer (KYN) service, and the like. Conversational AI engine 121 may be communicably engaged with external server 133 via at least one data transfer interface to execute one or more functions or operations for configuring, implementing and/or executing the conversational AI model and/or other functions (such as participant identity verifications).

In accordance with certain aspects of the present disclosure, participant user 22 may provide a voice input to smart speaker 102 or a microphone of participant client 104 to invoke one or more functions of conversational AI agent 122. A conversational input (e.g., voice or text) may be converted into a digital audio format and streamed to a communications module of application server 110. The communications module may be configured to provide a raw audio input to conversational AI agent 122 (e.g., in real-time). In accordance with certain aspects of the present disclosure, system 100 is configured to facilitate a plurality of conversational, multi-turn interactions between participant user 22 and conversational AI agent 122.

In accordance with certain aspects of the present disclosure, an exemplary use case of system 100 is initiated within investigator/sponsor environment 105. In accordance with certain embodiments, investigator user 24 may instantiate an investigator instance 120′ of clinical trial management application 120 at practitioner client 108. Investigator instance 120′ may comprise a graphical user interface configured to enable investigator user 24 to configure a plurality of parameters for the conduct and management of a clinical trial for an investigational medical intervention; e.g., in accordance with one or more clinical trial protocols (as described in more detail herein below). In certain embodiments, investigator instance 120′ may comprise a plurality of pre-populated data for participant user 22 comprising a plurality of health record data to assist investigator user 24 in configuring a plurality of clinical trial management parameters.

Investigator instance 120′ may be configured to communicate a plurality of user-generated data, sensor data, objective clinical data and the like to application server 110 via communications network 118. Application server 110 may receive and process the data according one or more data processing framework embodied in algorithmic logic engine 123. In accordance with certain embodiments, algorithmic logic engine 123 may be configured to process data in accordance with a plurality of configurable clinical trial management parameters and provide one or more outputs to conversational AI agent 122. Conversational AI agent 122 may comprise an AI framework comprising a neural network architecture configured to enable one or more automated speech recognition (ASR), natural language processing (NLP), natural language understanding (NLU), dialogue management, text-to-speech (TTS) converter function, speech-to-text (STT) converter function, or speech-to-speech (STS) function.

In accordance with certain aspects of the present disclosure, application server 110 may receive one or more objective clinical data inputs for participant user 22 via one or more of participant client 104, testing device 106, external EMR/EHR server 130 and/or clinical trial management server 114. Examples of objective clinical data may include, but are not limited to, basic metabolic panel (e.g., sodium, potassium, chloride, bicarbonate, BUN, creatinine, glucose, magnesium, phosphate, calcium, uric acid, and the like), hemoglobin A1C, medication adherence based on participant-reported data (e.g., prescription (Rx) fill data, log data, and other data sources), blood pressure data and other physiological sensor data, participant-reported side effects, and the like. In certain embodiments, participant-reported data may be received via a user interface 120″. Algorithmic logic engine 123 may receive and process the objective clinical data inputs and provide one or more outputs to conversational AI engine 121. Conversational AI agent 122 may be configured to generate and deliver one or more conversational prompts to participant user 22 via a conversational interface of participant client 104 or smart speaker 102. In accordance with certain aspects of the present disclosure, the one or more conversational prompts comprise one or more instructions, reminders, check-ins or other interaction associated with the clinical trial management protocol.

In accordance with certain aspects of the present disclosure, participant user 22 may provide a conversational input (e.g., voice or text) at smart speaker 102 or participant client 104 in response to the one or more conversational prompts. Algorithmic logic engine 123 may process the conversational input and, optionally, the objective clinical data (e.g., at one or more time points) and provide one or more outputs to conversational AI agent 122. In accordance with certain aspects of the present disclosure, conversational AI agent 122 may generate a second or subsequent generative voice prompt and output the second or subsequent generative voice prompt to participant user 22 via smart speaker 102 or participant device 104. In accordance with certain aspects of the present disclosure, the second or subsequent generative voice prompt comprises a second or subsequent reminder, check-in or other interaction for participant user 22 according to one or more clinical trial management protocol.

In accordance with certain exemplary embodiments of the present disclosure, system 100 is configured to facilitate one or more informed consent (eConsent) and onboarding protocols for a remote clinical trial for an investigational medical intervention. In certain embodiments, system 100 is configured to facilitate collection of remote informed consent by participant user 24 via one or more system routines. In accordance with certain embodiments, clinical trial management application 120 may comprise an invitation module configured to send participant user 22 a single-use, cryptographically signed deep-link. Participant user 22 may open the link at user interface 120″ and launch a guided onboarding sequence. In certain embodiments, the onboarding sequences may comprise capturing a live selfie of participant user 22 beside a government-issued ID at participant client 104, relaying the images to an external KYC-liveness service, and storing the returned “Verified-ID” token at a local storage of participant client 104. Next, clinical trial management application 120 may present a current IRB-approved consent form (e.g., pulled directly from a version-controlled repository of application database 112) as a scrollable HTML/PDF with glossary pop-ups and an embedded comprehension quiz for participant user 22 to complete. If participant user 22 quiz score falls short of a predefined threshold, participant user 22 is automatically encouraged to review the material again before proceeding. In certain embodiments, clinical trial management application 120 may be configured to send a one-time passcode to participant client 104 to confirm possession of the device by participant user 22. User interface 120″ may be configured to present an electronic signature field to participant user 22 for electronic signature. When the signature is applied, application server 110 may be configured to hash the precise consent-form version, the signature image, the Verified-ID token, the quiz score, and the OTP proof, and commit that bundle as an immutable entry in an audit ledger at application database 112. Application server 110 may return a short-lived onboarding token to mark participant user as an enrolled participant. In accordance with certain embodiments, every subsequent API call from participant client 104 must present this token, and each use is independently logged to the audit ledger. This architecture and framework provides clinical trial stakeholders with end-to-end proof that every remote participant has been positively identified, fully informed, and legally consented before any study activity begins.

In accordance with certain exemplary embodiments of the present disclosure, system 100 is configured to facilitate one or more participant eligibility screening for a remote clinical trial for an investigational medical intervention. In accordance with certain embodiments, application server 110 is configured to send a secure, single-use link that opens a screening workspace inside user interface 120″ to a candidate user (prior to onboarding, participant user 22 may be referred to as a candidate user). The screening workspace may comprise an adaptive questionnaire configured to collect demographics, medical history, and concomitant medications from the candidate user. In certain embodiments, sensor device 109 may collect/stream physiological measurements (e.g., baseline heart-rate, oxygen saturation, or glucose data) to application server 110 in real time. If the eligibility protocol calls for additional evidence, such as recent laboratory values or imaging, the candidate user can upload scanned documents or photographs via user interface 120″, which may be immediately encrypted and hashed for integrity.

In accordance with certain embodiments, while questionnaire is under way, a real-time audio/video channel may connect the candidate user to a remote clinician to perform a brief tele-physical exam. System 100 may comprise a remote clinician interface configured to enable the clinician to enter observations, order lab tests and view candidate data. Application server 110 may comprise a data aggregator configured to normalize formats and timestamps for incoming data flows before passing a consolidated record to an eligibility-criteria engine. The eligibility-criteria engine may apply the sponsor's inclusion and exclusion rules, lab ranges, and safety thresholds for the remote clinical trial and issue an eligibility verdict. In certain embodiments, system 100 may comprise a clinician override panel configured to enable a principal investigator to approve borderline cases or screen-fail candidates with a cryptographically signed justification. In accordance with certain aspects of the present disclosure, every decision, automated or human, generates an immutable ledger entry that chains to prior records to enable regulators/reviewers to reconstruct how each participant was qualified. Candidates who pass may receive a time-bound, digitally signed “screen-pass” token. This token must be presented, and is independently logged, when the participant proceeds to eConsent, dosing prompts, or any subsequent study activity to ensure that only properly screened individuals enter the clinical trial workflow.

In accordance with certain aspects of the present disclosure, system 100 is configured to facilitate one or more remote dosing instructions and reminders for an investigational medical intervention. In accordance with certain aspects of the present disclosure, participant environment 103 is configured as a compliant dosing site via the orchestration of scheduling, reminders, and confirmation loops facilitated via application computing environment 101. In accordance with certain embodiments, algorithmic logic engine 123 is configured to execute a dosing protocol.

Clinical trial management application 120 may comprise a scheduling service configured to calculates an exact administration window (e.g., taking time-zone, prior adherence, and any active safety lockouts into account) and packages that information into an encrypted message (e.g., voice and/or text) for participant user 22. In accordance with certain embodiments, conversational AI agent 122 may deliver a generative voice prompt and/or on-screen instructions to participant user 22.

In accordance with certain embodiments, if participant user 22 fails to acknowledge the prompt or instructions within a grace period, conversational AI agent 122 may escalate one or more follow-up prompts and/or facilitate a phone-based voice prompt via a voice-over-IP (VOIP) system. When the dose is taken, participant user 22 may confirm either by speaking a short phrase, typing a short phrase, scanning a pack barcode, or tapping a graphical element within user interface 122″. Application server 110 hashes the confirmation, stamps it with the exact timestamp and device signature, and records it in the immutable audit ledger at application database 112 (e.g., alongside any concurrent sensor data that might corroborate ingestion; for example, a heart-rate uptick or accelerometer motion from a connected sensor).

In accordance with certain aspects of the present disclosure, each confirmation feeds back to a compliance monitor module, which continuously recalculates an adherence score. High adherence quietly advances the trial schedule, while skipped or late events raise a deviation risk for participant user 22. In such cases, clinical trial management application 120 may comprise a reflex logic to trigger tailored follow-ups or, if thresholds are breached, an automated dose hold that must be cleared by investigator user 24. In accordance with certain aspects of the present disclosure, every prompt, reminder, acknowledgment, and adaptive adjustment is cryptographically chained to prior records, giving sponsors and regulators real-time, tamper-evident proof that doses are administered in accordance with clinical trial protocols.

In accordance with certain aspects of the present disclosure, system 100 is configured to facilitate the collection of one or more participant-reported outcome (ePRO) diaries. In accordance with certain embodiments, clinical trial management application 120 comprises a back-end scheduler configured to calculate a periodic or event-driven “diary window” for participant user 22. In accordance with certain embodiments, the diary window is configured to drive one or more generative prompts from conversational AI agent 122 to participant user 22 (e.g., via participant client 104 and/or smart speaker 102). Raw voice may be routed through an audio-processing module at application server 110, where it is transcribed, sentiment-scored, and merged with structured answers before the combined record is hashed and committed to the immutable ledger at application database 110.

If a diary is missed, algorithmic logic engine 123 may comprise a reflex logic to escalate one or more follow-ups by conversational AI agent 122. A compliance module may be configured to recalculate an adherence metric that feeds into a deviation-prevention logic and, when necessary, trigger the follow-up prompts or investigator alerts. Each diary entry may be scored in real time. Scores may flow directly into an adaptive-dosing engine and/or a safety-signal engine. This enables system 100 to provide a continuous, participant-reported pulse on efficacy and tolerability to investigator user 24.

In accordance with certain aspects of the present disclosure, system 100 is configured to facilitate real-time safety monitoring and adverse event (AE) triage for participant user 22. In accordance with certain embodiments, system 100 is configured to enable continuous, virtual observation of participant user 22 by streaming raw sensor feeds, voice-derived psychophysiological indicators, and diary scores into a low-latency data bus residing on application server 110. In accordance with certain embodiments, data fragments are time-aligned and merged into a participant-specific stream and inspected by an analytics layer. The analytics layer may be configured to looks for negative leading indicators; for example, an SpO₂ dip, an abrupt rise in ePRO symptom grade, a vocal stress pattern that historically tracks with nausea or dizziness, and the like. When the analytics layer detects an anomaly, it may apply a protocol-driven safety matrix. For example, “green” events may be logged in the background; “amber” events may trigger an immediate follow-up prompt that asks clarifying questions while routing the data to investigator interface 120′; and “red” events may flip an automated dose-lockout flag, invoke an on-call nurse via a secure video, and/or push an alert payload to investigator client 108. In accordance with certain aspects of the present disclosure, all data is written to an immutable row in the audit ledger and is cryptographically chained to prior records so the entire safety narrative can be reconstructed. If a clinician clears an alert, the signed resolution propagates back through the same chain. In said embodiments, the lockout may be lifted only after the ledger entry is sealed. By fusing multi-modal signals, automated analytics, and human oversight inside a tamper-evident framework, system 100 delivers real-time pharmacovigilance designed to mimic in-clinic monitoring.

In accordance with certain aspects of the present disclosure, system 100 is configured to facilitate adaptive dose-escalation within adaptive-design trials. In accordance with certain embodiments, algorithmic logic engine 123 comprises an adaptive decision layer that may employ any of a variety of statistical or computational frameworks, including without limitation Bayesian models, frequentist or likelihood-based designs, model-assisted or model-free adaptive algorithms, rules-based escalation schemes, reinforcement-learning controllers, fuzzy-logic systems, or combinations thereof. In accordance with certain embodiments, algorithmic logic engine 123 evaluates accumulating subject data together with protocol-defined constraints to recommend dose-escalation, de-escalation, cohort expansion, arm suspension, or other adaptive trial actions. In certain embodiments, after every dosing cycle, algorithmic logic engine 123 analyzes participant-reported data, physiological sensor data, laboratory and/or testing device data, and psychophysiological voice-markers. The plurality of data sets are continually incorporated into dose-toxicity curve. A posterior probability is continuously recalculated such that each prescribed level will exceed the protocol's target toxicity threshold. The model is thereby configured to output a decision to escalate, hold, or de-escalate a next dose of an investigational medical intervention. Such decision may be tempered by individual-level safety gates, such as cumulative dose ceilings, dose-limiting-toxicity flags, and clinician overrides, which can force a dose hold or trigger an investigator alert before the next prompt is transmitted. Once cleared, conversational AI agent 122 may be configured to communicate a generative voice prompt comprising the new instruction to participant user 22 (e.g., via participant client 104 or smart speaker 102). All data (e.g., raw data hashes, posterior statistics, rule identifiers, and sent prompt IDs) may be immutably chained to the audit ledger at application database 112.

In accordance with certain aspects of the present disclosure, system 100 is configured to facilitate virtual visits in clinical trials. In accordance with certain embodiments, system 100 is configured to collapse a traditional on-site visit into a sequence of coordinated digital touchpoints, allowing sponsors to run fully virtual or hybrid trials without sacrificing the depth of data normally gathered in a clinic. At each scheduled “visit window,” application computing environment 101 pushes a secure bundle of tasks to participant user 22 (e.g., via participant client 104 or smart speaker 102). In certain embodiments, the tasks may comprise a real-time video link to a clinician, an at-home vitals capture routine driven by connected sensors, an interactive questionnaire that mirrors the protocol's case-report forms, and optional sample-collection prompts supported by barcoded mail-back kits. As participant user 22 completes each step, encrypted audio, video, sensor packets, and form data may be streamed from participant computing environment 103 to application server 101. A time-sync layer may be configured to assemble said data into a visit record that meets the same source-document standards as an onsite encounter. Clinical trial management application 120 may comprise one or more intelligent checklists configured to ensure that mandatory components (e.g., eye-contact verification during video, device serial numbers on sensor uploads, tamper-evident seals on kit scans) are satisfied before the visit is marked complete. Any missing element may trigger an automated reminder loop and, if unresolved, escalate to a coordinator alert to prevent protocol deviations in real time. Once the record passes validation, application server 110 stamps the record with a cryptographic hash and chains it into the immutable audit ledger. A scheduling engine may be updated to maintain a sync between downstream dosing, diary prompts, and laboratory logistics. This closed-loop workflow enables hybrid trials between remote and in-person touchpoints.

In accordance with certain aspects of the present disclosure, system 100 is configured to facilitate a data pipeline in accordance with one or more regulatory-compliant data formats (e.g., EDC or CTMS). In accordance with certain aspects of the present disclosure, system 100 streams every validated data point (e.g., voice-prompt transactions, sensor readings, diary scores, safety signals, kit bar-codes, and investigator notes) into a canonical event bus. Those events are then mapped to an electronic case report form (eCRF) schema. In certain embodiments, an integration layer watches the bus for new ledger-sealed rows. Protocol metadata may be added to the ledger-sealed rows. The data may be transformed into CDISC-compliant payloads that can traverse sponsor firewalls without exposing the raw, participant-identifiable artifacts. In certain embodiments, signed JSON bundles may flow through a zero-trust API gateway into investigator/sponsor environment 105. Operational milestones (e.g., screen-pass issued, dose cohort advanced, deviation adjudicated) may feed parallel message queues that keep the CTMS aligned with participant status and site workload. Because every outbound packet carries the hash of its source ledger entry, investigator user 24 can trace any value in the EDC back to the exact prompt, timestamp, and device signature that generated it, and auditors can replay the entire chain end-to-end without parsing separate logs.

In accordance with certain aspects of the present disclosure, system 100 readily extends beyond the controlled trial period into the post-market phase. Because application computing environment 101 stores every study workflow as reusable protocol parameters, a sponsor can load an “approved-product” schema that substitutes formal visit schedules with longitudinal engagement cadences and augments a safety-threshold library with post-marketing reporting rules (e.g., 15-day serious adverse-event deadlines). Investigator/sponsor environment 105 thus pivots from supervising dose escalation to overseeing real-world product performance while participant environment 103 continues to serve as a distributed data-collection network.

During commercial use, participant devices, connected wearables, and in the case of combination products, smart packaging stream physiologic signals, usage telemetry, and barcode-encoded lot identifiers through the same secure channel to application server 110. An analytics layer running inside algorithmic logic engine 123 screens the aggregated feed for unexpected frequency, severity, or clustering of events and raises graded alerts in near-real time. When an event crosses a configurable signal-detection boundary, system 100 automatically drafts an individual case safety report (ICSR), links it to the immutable audit hash, and pushes it to the sponsor's pharmacovigilance module or directly to external databases such as FAERS or EUDRAVIGILANCE (e.g., all without breaking the cryptographic chain of custody preserved in the audit ledger).

In accordance with certain aspects of the present disclosure, conversational AI agent 122 keeps patients and caregivers engaged after market entry by delivering periodic voice or text prompts that collect device-handling feedback, over-the-counter co-medication details, and quality-of-life metrics. The diary engine dynamically widens or narrows questioning based on sentiment trends or sensor-detected anomalies, ensuring that the sponsor captures context-rich, patient-reported outcomes that complement objective telemetry. In accordance with certain embodiments, all post-market interactions (e.g., prompts, audio files, sensor packets, clinician adjudications, and automated ICSRs) enter the same tamper-evident ledger used during the clinical phase, giving regulators an unbroken, inspection-ready record from first-in-human dosing through commercial distribution. In certain embodiments, investigator interface 120′ may allow safety scientists to slice real-world evidence by geography, batch, or device firmware version, and to trigger targeted recall notices or field-safety corrective actions through the secure messaging backbone within minutes of issue confirmation.

In accordance with certain aspects of the present disclosure, system 100 can be tuned to harvest high-fidelity real-world evidence (RWE) while the trial is still in progress. In accordance with certain embodiments, participant environment 103 streams continuous physiologic and behavioral signals (e.g., heart-rate variability, actigraphy, geolocation, glucose, and other wearable metrics) through sensor device 109 and participant client 104 into application server 110, where each packet is time-synced and merged with conversational diary data and site-entered clinical results. Application-computing environment 101 also maintains secure interfaces to external EMR/EHR server 130 and other third-party data sources. These APIs allow the algorithmic logic engine 123 to enrich in-trial observations with longitudinal lab records, pharmacy-dispensing events, and payer or registry feeds, transforming the controlled study into a living laboratory that mirrors real-world use without sacrificing data provenance.

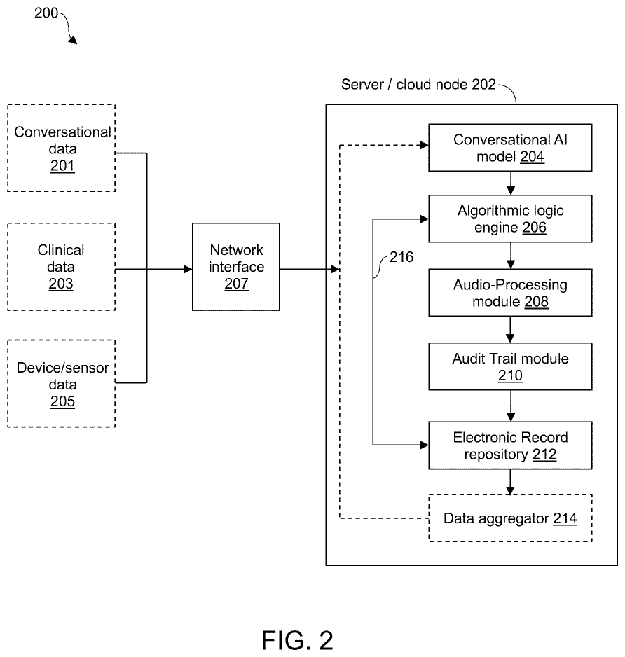

Inside application server 110, data aggregator 214 (as shown in FIG. 2) normalizes heterogeneous payloads and writes every validated item (e.g., sensor readings, diary transcripts, voice-stress scores, EHR imports) onto a canonical event bus. An integration layer watches the bus, maps each event to a CDISC-compliant schema, and publishes de-identified packets into investigator/sponsor environment 105 so biostatisticians can run interim analyses that combine randomized trial outcomes with real-world utilization patterns. Because every outbound packet carries the hash of its source ledger row, regulators can trace an aggregate endpoint (e.g., such as device-free days or glucose-time-in-range) back to the exact prompt, sensor, timestamp, and EHR record that generated it. This immutable chain of custody supplies inspection-ready evidence that the AI-driven workflows produce source-document-equivalent RWE, enabling sponsors to support label expansions, health-technology-assessment dossiers, and adaptive-licensing submissions directly from the ongoing study.

Referring now to FIG. 2, a functional block diagram of a conversational system 200 for automated remote management of clinical trials is shown. In accordance with certain aspects of the present disclosure, system 200 may comprise an embodiment of system 100, as shown in FIG. 1. In accordance with certain aspects of the present disclosure, system 200 comprises an integrated computing environment that orchestrates conversational interactions with trial participants, ingests heterogeneous study data, and produces protocol-compliant outputs for sponsors, investigators, and regulators to enable compliant, automated remote management of clinical trials. In accordance with certain embodiments, system 200 comprises an ingress layer comprising multi-modal data sources. In certain embodiments, the multi-modal data sources comprise conversational data 201, clinical data 203 and device/sensor data 205. Conversational data 201 includes raw or pre-parsed utterances, transcripts, chat messages, and associated metadata (e.g., timestamps, speaker IDs, language tags) originating from a participant client (e.g., participant client 104 as shown in FIG. 1). Clinical data 203 may comprise structured and unstructured information from electronic health records (EHR), laboratory information systems (LIS), and third-party electronic data-capture (EDC) platforms. Device/sensor data 205 comprises physiologic or behavioral signals (e.g., heart-rate variability, actigraphy, geolocation, blood pressure, blood glucose measurements, heart rate, etc.) generated by wearables, IoT medical devices, or smartphone sensors. Each feed may be streamed or batch-uploaded through a network interface 207 into cloud node/server 202. Network interface 207 may comprise an API gateway, message-queue broker, or secure WebRTC tunnel. Server/cloud node 202 may comprise application server 110 as shown in FIG. 1.

In accordance with certain aspects of the present disclosure, server/cloud node 202 comprises a cloud execution layer comprising a plurality of micro-services. The plurality of micro-services may comprise a conversational AI model 204, an algorithmic logic engine 206, an audio-processing module 208, an audit trail module 210, an electronic record repository 212, and a data aggregator 214. In accordance with certain embodiments, conversational AI model 204 comprises a large-language model or dialogue-management model that is fine-tuned on clinical corpora. Conversational AI model 204 is configured to perform intent recognition, named-entity extraction, sentiment scoring, and response generation in real time. Audio-processing module 208 may be configured to apply signal-conditioning (e.g., denoise, voice-activity detection, etc.), feature extraction (e.g., MFCCs, prosodic vectors, etc.), and, when appropriate, vocal-biomarker inference before passing embeddings to conversational AI model 206 or downstream analytics. Algorithmic logic engine 206 may be configured to execute study-specific business rules such as eligibility gating, dose-escalation decisions, protocol-deviation alerts, and dynamic scheduling of participant tasks. In certain embodiments, algorithmic logic engine 206 may be configured to consume outputs from both conversational AI model 206 and the aggregated clinical/device data streams. In accordance with certain embodiments, audit-trail module 210 records an immutable, timestamped ledger of every system event (e.g., dialogue turns, model inferences, rule triggers, and database transactions) to satisfy 21 CFR Part 11, GDPR, and HIPAA traceability mandates. Electronic-record repository 212 provides durable object storage (e.g., S3, distributed SQL) for consent forms, eCRFs, annotated transcripts, and sensor payloads. In accordance with certain embodiments, electronic-record repository 212 is configured to enable role-based access controls and versioning. Data aggregator 214 normalizes heterogeneous schemas, resolves participant identifiers, and time-syncs multimodal observations into a canonical event ledger that algorithmic logic engine 206 can query with low latency via an egress/integration layer 216. In accordance with certain aspects of the present disclosure, one or more processed outputs of egress/integration layer 216 may include structured JSON or HL7 FHIR bundles pushed to sponsor EDC/CTMS systems; real-time REST/WebSocket callbacks that update mobile apps with next-best actions, reminders, or adaptive questionnaires; and analytics dashboards and regulatory submissions (e.g., PDF/CSV exports of the audit trail module 210).

While FIG. 2 illustrates the components of system 200 in a single tenancy, individual modules (e.g., 204, 206, 208, etc.) can be containerized and auto-scaled across multiple availability zones for fault tolerance. In certain embodiments, network interface 207 supports mutual-TLS, OAuth 2.0, and token-based device attestation to comply with zero-trust security principles. In accordance with certain embodiments, system 200 comprises an agnostic architecture configured to facilitate modality expansions; for instance, additional data feeds (e.g., imaging, genomics) can register with the data aggregator 214 without refactoring core services. In accordance with certain aspects of the present disclosure, server/cloud node 202 is configured to transform raw conversational and physiologic signals into compliant electronic records and study decisions, to enable fully remote and adaptively managed clinical trials.

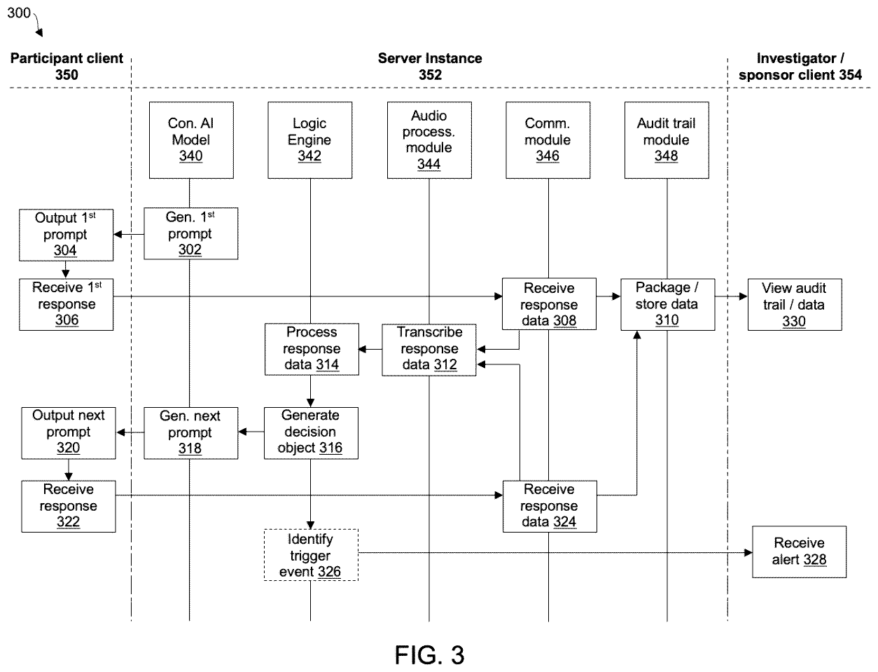

Referring now to FIG. 3, a process flow 300 of a conversational system and method for automated remote management of clinical trials is shown. In accordance with certain aspects of the present disclosure, the conversational system and method for automated remote management of clinical trials may comprise system 100 as shown and described in FIG. 1. One or more steps or operations of process flow diagram 300 may be embodied as one or more operations of system 100 as shown in FIG. 1 and/or system 200 as shown in FIG. 2. In accordance with certain aspects of the present disclosure, process flow 300 comprises a runtime interaction sequence for a conversational remote-trial session. In accordance with certain aspects of the present disclosure, process flow 300 comprises an exemplary sequence of operations 302-328 that occur during one conversational “turn loop” between a participant client 350, an elastic server instance 352, and an investigator/sponsor client 354. In accordance with certain embodiments, participant client 350 may comprise participant client 104 as shown in FIG. 1. Elastic server instance 352 may comprise an instance of application server 110 as shown in FIG. 1. Investigator/sponsor client 354 may comprise investigator client 108 as shown in FIG. 1. In accordance with certain embodiments, server instance 352 encloses five cooperating micro-services comprising a conversational AI model 340, an algorithmic logic engine 342, an audio-processing module 344, communications module 346, and audit-trail module 348. Micro-services 340-348 may be jointly configured to transform raw utterances (e.g., from a participant user) into protocol-compliant decisions, alerts, and immutable records.

In accordance with certain aspects of the present disclosure, process flow 300 may comprise one or more steps or operations to initiate a multi-turn conversational interaction between a conversational AI agent and a participant user (e.g., via a participant client device). In accordance with certain embodiments, conversational AI model 340 is configured to generate a first prompt (step 302) tailored to the participant's schedule, language, and dose cohort. The first prompt is output to the participant device (step 304) as text or synthesized speech. A verbal or typed reply by the participant user is captured by the participant client (step 306) and forwarded to the server (e.g., via communications module 346) as a packet of response data 308 that may contain audio, timestamps, and device telemetry. Within the server, audio processing module 344 transcribes or feature-encodes the response data (step 312). Conversational AI model 340, in concert with algorithmic logic engine 342, processes the response (step 314) to extract intents, biomarkers, sentiment, and adverse-event cues. In accordance with certain embodiments, parsed content and metadata are packaged and stored in secure object storage (step 310), while the audit-trail module 348 contemporaneously logs every transaction with a cryptographic timestamp.

In accordance with certain aspects of the present disclosure, process flow 300 may comprise one or more steps or operations for generating a decision object and generating a follow-up prompt. In accordance with certain embodiments, logic engine 342 generates a decision object (step 316) (e.g., “advance to 20 mg,” “issue adherence reminder,” “flag possible Grade 2 AE”) according to one or more study-specific rules (e.g., dose-escalation, eligibility gating, or schedule optimization). Based on the decision object (step 316), conversational AI model 340 produces the next prompt (step 318), which the communications module immediately delivers to the participant client (step 320), which in turn initiates the next response loop at step 322 and step 324.

In accordance with certain aspects of the present disclosure, if algorithmic logic engine 342 detects a trigger event (step 326) (e.g., such as a protocol deviation, safety signal, or unblinded efficacy threshold), algorithmic logic engine 342 instructs communications module 346 to push an alert (step 328) (e.g., SMS, in-app push, generated voice, or email) to the investigator/sponsor client 354. In accordance with certain embodiments, investigators can view a real-time audit trail or underlying data (step 330) through a secure dashboard to facilitate rapid oversight, serious adverse events (SAE) adjudication, or data and safety monitoring board (DSMB) queries. In accordance with certain aspects of the present disclosure, each arrow in process flow 300 corresponds to an API call, message-queue post, or WebRTC stream secured with mutual-TLS and token-based device attestation. In accordance with certain aspects of the present disclosure, audit trail entries from audit trail module 348 satisfy 21 CFR Part 11, GDPR, and HIPAA by recording user IDs, data-hash digests, and time-sync offsets. Although presented as a linear sequence, steps 308-316 may be executed asynchronously in a containerized pipeline, allowing the architecture to auto scale across availability zones and maintain sub-second latency for multi-lingual, geographically dispersed clinical trials. By coordinating the foregoing enumerated operations, process flow 300 converts unstructured conversational input into structured electronic records, protocol decisions, and regulatory-grade audit artifacts to enable enabling safe, adaptive, and fully remote clinical trials.

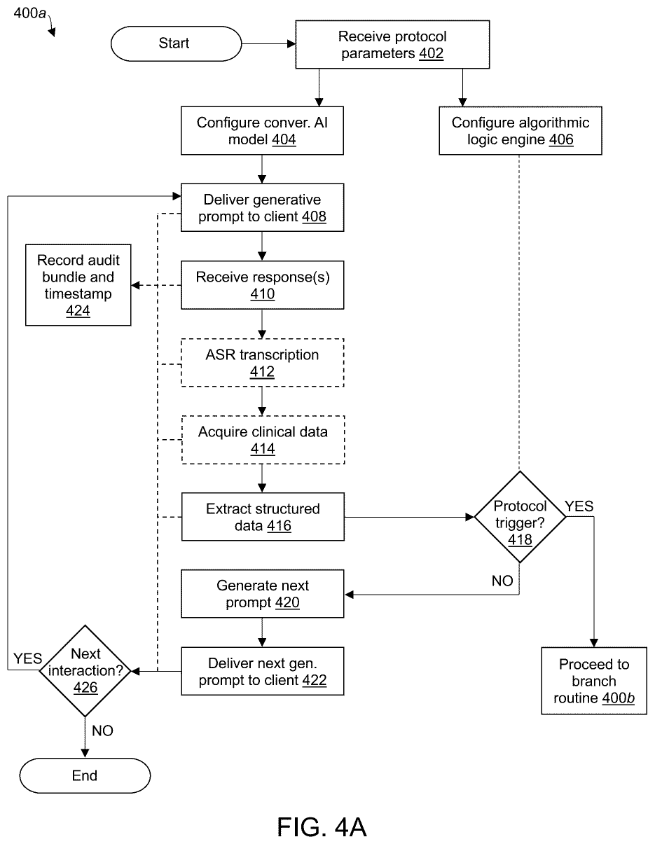

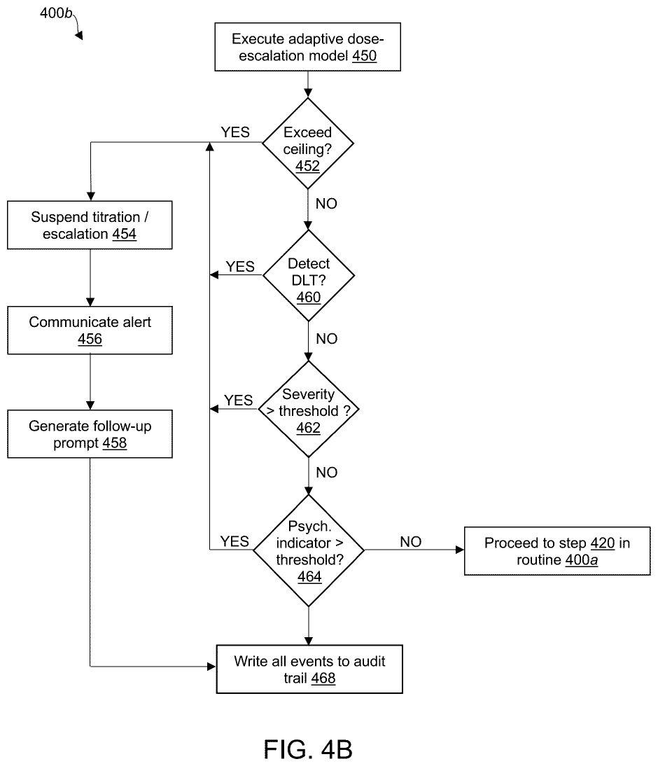

Referring now to FIG. 4A, a logic flow diagram of a routine 400a of a conversational system for automated remote management of clinical trials. In accordance with certain aspects of the present disclosure, the conversational system for automated remote management of clinical trials may comprise system 100 as shown and described in FIG. 1. One or more steps or operations of routine 400a may be embodied as one or more operations of system 100 as shown in FIG. 1 and/or system 200 as shown in FIG. 2. The operations in routine 400a may be performed in the order presented, in a different order, or simultaneously. Further, in some exemplary embodiments, some of the operations may be omitted, added, modified, skipped, or the like without departing from the scope of the invention. In accordance with certain embodiments, routine 400a comprises steps 402-426 for a steady-state conversational loop between a conversational AI agent and a participant user.

In accordance with certain aspects of the present disclosure, routine 400a comprises one or more steps or operations for receiving one or more parameters for a clinical trial protocol via an investigator or sponsor user at an investigator or sponsor interface (Step 402). In accordance with certain embodiments, a cloud service ingests a current protocol version, visit cadence, dose windows, language, and cohort identifiers from an electronic-trial-master-file (eTMF) or EDC API. These parameters are cached in session scope for deterministic execution. Routine 400a may further comprise one or more steps or operations for configuring a conversational model (step 404) and configuring an algorithmic logic engine (step 406). In accordance with certain embodiments, step 404 comprises one or more steps or operations for configuring a large-language model or finite-state dialogue model based on the protocol parameters received at step 402. Step 404 may comprise one or more steps or operations for constraining intent recognition and generation to protocol-sanctioned utterances. In accordance with certain embodiments, at step 406, study-specific rule graphs (e.g., eligibility gates, adverse-event severity thresholds, adherence windows) may be loaded into memory to enable constant-time rule evaluation during the conversational session with the participant user. Routine 400a may proceed by executing one or more steps or operations for delivering a generative prompt to a participant client (e.g., via the conversational AI agent) (step 408). In accordance with certain embodiments, at step 408, the server streams (e.g., via a communication module) an initial prompt (e.g., voice or text) to the participant device, personalized for locale, visit number, and dosing schedule. Routine 400a may proceed by executing one or more steps or operations for receiving one or more conversational responses from the participant user (e.g., via the participant client) (step 410). In accordance with certain embodiments, at step 410, the one or more conversational responses may comprise typed chat, microphone audio, or hybrid payloads. The one or more conversational responses may be communicated to the application server via a mutually-authenticated REST/WebRTC channels, bundled with timestamps, device ID, locale, and network QoS metadata. Where the one or more conversational responses comprise a voice response, routine 400a may comprise one or more automated speech recognition transcription operations (step 412). For audio payloads, an automatic-speech-recognition pipeline converts speech to text while preserving word-level time stamps for downstream vocal-biomarker analysis. In certain embodiments, routine 400a may comprise one or more steps or operations for acquiring objective clinical data (e.g., via one or more clinical endpoints) (step 414). In said embodiments, step 414 comprises one or more steps or operations for querying participant vitals, lab values, or wearable telemetry so that conversational context is enriched with objective physiologic evidence. Routine 400a may proceed by executing one or more steps or operations for extracting structed data from the conversational responses and objective clinical data (step 416). In certain embodiments, a feature-extraction service performs named-entity recognition, intent parsing, sentiment scoring, and biometric feature generation, yielding a canonical JSON event object keyed on the participant's GUID and session time. The extracted structed data may be passed to the algorithmic logic engine. A reflex logic of the algorithmic logic engine may be configured to determine whether a threshold value for a protocol trigger is present in the structured data (step 418). If YES, routine 400a proceeds to branch routine 400b (as shown in FIG. 4B). If NO, routine 400a proceeds to generate the next generative prompt (e.g., via the conversational AI model) (step 420). Adaptive logic may randomize PRO items or inject adherence nudges. The next generative prompt is delivered to the participant client (e.g. via the communications module), beginning the next dialogue turn on the participant device (step 422). Routine 400a may comprise a decision step 426 to determine whether the trial protocol warrants a next interaction with the participant user. If YES, the conversation loop continues at step 408. If NO, routine 400a concludes. In accordance with certain aspects of the present disclosure, all raw payloads, model outputs, rule evaluations, and network receipts are hashed and persisted to an immutable ledger that satisfies 21 CFR Part 11, GDPR, and HIPAA traceability mandates (step 424).

Referring now to FIG. 4B, a logic flow diagram of a branch routine 400b of the system for automated remote management of clinical trials. In accordance with certain aspects of the present disclosure, routine 400b is entered only when decision diamond 418 in FIG. 4A evaluates to YES. Routine 400b orchestrates adaptive dosing logic, safety monitoring, psychological-indicator thresholds, and sponsor/investigator alerting before returning control to step 420 in FIG. 4A in the steady-state loop.

In accordance with certain aspects of the present disclosure, routine 400b comprises one or more steps or operations for executing an adaptive dose-escalation model (step 450) via the algorithmic logic engine. The adaptive dose-escalation model may comprise a Bayesian continual-reassessment (mTPI) or CRM module configured to compute a recommended next dose level (e.g., “escalate,” “hold,” “de-escalate”) using accumulated toxicity and efficacy data. In accordance with certain embodiments, the algorithmic logic engine sums the participant's cumulative dispensed dose and compares it to a protocol-defined limit to determine whether a ceiling has been exceeded (step 452). If YES, further dose increases are halted; the current dose instruction is flagged as “hold,” and a safety note is appended to the participant's record (step 454). If NO, the algorithmic logic engine inspects real-time adverse-event classifiers and sensor streams for a dose-limiting-toxicity signature (step 460). If YES, further dose increases are halted at step 454. If NO, the algorithmic logic engine proceeds to analyze a severity threshold for the dose-limiting-toxicity (step 462). If the dose-limiting-toxicity exceeds the severity threshold, routine 400b proceeds to step 454. If the dose-limiting-toxicity does not exceed the severity threshold, the algorithmic logic engine proceeds to analyze whether a phycological indicator exceeds a predetermined threshold value (step 464). In certain embodiments, step 464 comprises one or more steps or operations for comparing vocal or textual sentiment, stress, or depression markers to one or more risk thresholds. If the phycological indicator does not exceed the predetermined threshold value, routine 400b joins routine 400a at step 420. If the phycological indicator does exceed the predetermined threshold value, routine 400b proceeds to step 454 to suspend titration or escalation of the participant's current dose. In certain embodiments, the communications module dispatches real-time notifications (e.g., e-mail, SMS, dashboard push) to the medical monitor, investigator of record, and DSMB, with the relevant audit hash embedded for traceability (step 456). Irrespective of prior branching, the conversational AI model crafts a follow-up prompt reflecting the updated dosing plan or safety instructions (e.g., “Please pause your next dose and await a call from the study nurse”) (step 458). In accordance with certain aspects of the present disclosure, every branch decision, model inference, and outbound alert is atomically committed to the immutable ledger with cryptographic timestamps. In accordance with certain aspects of the present disclosure, routines 400a and 400b comprise steps and operations for converting unstructured conversational input and multimodal clinical evidence into adaptive prompts, dose-management decisions, and regulator-grade audit artifacts to enable safe, compliant, fully remote clinical trials.

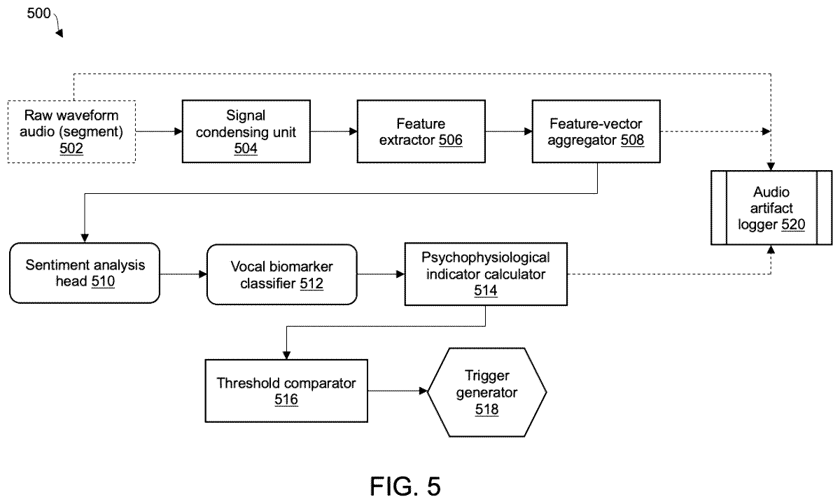

Referring now to FIG. 5, a process flow diagram of an audio processing routine 500 for the conversational system for automated remote management of clinical trials is shown. In accordance with certain aspects of the present disclosure, the conversational system for automated remote management of clinical trials may comprise system 100 as shown and described in FIG. 1. One or more steps or operations of routine 500 may be embodied as one or more operations of system 100 as shown in FIG. 1 and/or system 200 as shown in FIG. 2. The operations in routine 500 may be performed in the order presented, in a different order, or simultaneously. Further, in some exemplary embodiments, some of the operations may be omitted, added, modified, skipped, or the like without departing from the scope of the invention. In accordance with certain embodiments, routine 500 comprises a modular audio-processing pipeline that transforms a short segment of raw microphone data into structured psychophysiological indicators and real-time safety triggers.

In accordance with certain aspects of the present disclosure, routine 500 comprises one or more steps or operations for receiving a raw waveform audio segment at an audio processing module (step 502). A time-domain slice (e.g., 3-10 seconds) of 16- to 24-bit PCM data may be captured by the participant device. The segment may be framed and passed downstream with its original sampling rate metadata. The raw waveform audio segment may be passed to a signal condensing unit 504. Condensing unit 504 may be configured to perform one or more steps or operations for performing denoising, A-weighting, automatic gain control (AGC), and voice-activity detection (VAD) on the raw waveform audio segment. In accordance with certain embodiments, non-speech regions may be discarded or down-sampled to reduce compute load and storage. In certain embodiments, a feature extractor 506 converts each speech frame into multi-modal acoustic features, such as 13-dimensional MFCCs, prosodic statistics (fundamental frequency, jitter, shimmer), and spectral roll-offs. A feature vector aggregator 508 stacks the frame-level feature vectors into fixed-length tensors (e.g., 256×T) using sliding-window or attention-pooling strategies, thereby capturing supra-segment dynamics such as intonation contours. In accordance with certain embodiments, a sentiment analysis head 510 comprises a light-weight transformer or convolutional neural network (CNN) that predicts continuous affect scores (e.g., valence, arousal) and categorical sentiment labels (e.g., positive, neutral, negative) from the aggregated tensor. An output of sentiment analysis head 510 is passed to a vocal biomarker classifier 512. Vocal biomarker classifier 512 may comprise a fine-tuned classifier that maps acoustic embeddings to clinical risk factors (e.g., fatigue, depression, anxiety, or respiratory impairment) using a multi-task loss to share signal representations with sentiment analysis head 510. A psychophysiological indicator calculator 514 combines outputs from sentiment analysis head 510 and vocal biomarker classifier 512 with contextual metadata (e.g., language, locale, time of day) to derive scalar indicators such as stress index, emotional volatility, or respiratory effort score. In accordance with certain embodiments, a threshold comparator 516 is configured to compare each indicator to study-specific safety and adherence thresholds (e.g., configurable via protocol parameters). If the study-specific safety and adherence thresholds are exceeded, a threshold generator 518 may produce a Boolean trigger flag. In accordance with certain embodiments, an audio artifact logger 520 is configured to persists raw waveforms, intermediate features, model logits, and trigger decisions with cryptographic hashes and timestamps to satisfy 21 CFR Part 11 audit requirements.

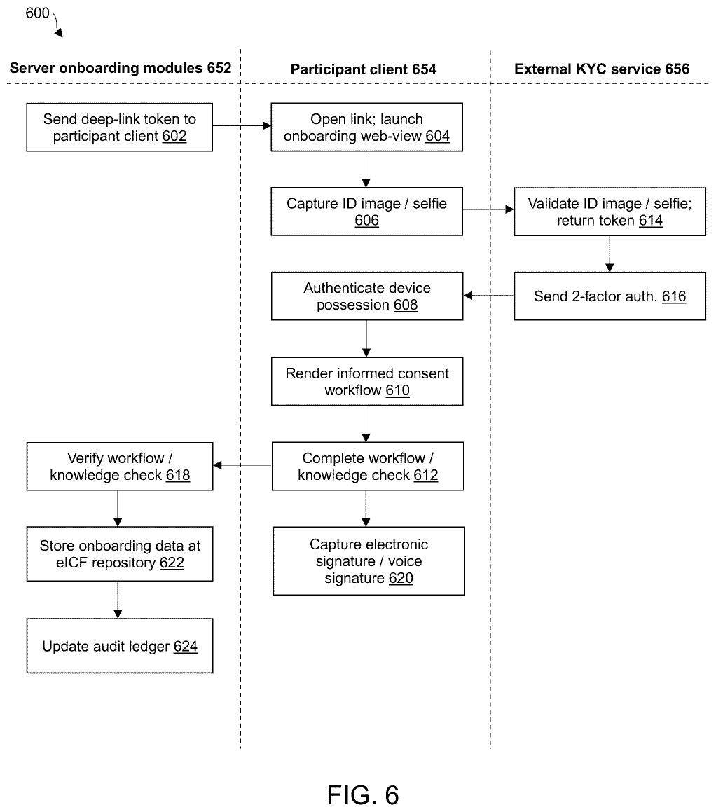

Referring now to FIG. 6, a logic flow diagram of a routine 600 of the system for automated remote management of clinical trials. In accordance with certain aspects of the present disclosure, the conversational system for automated remote management of clinical trials may comprise system 100 as shown and described in FIG. 1. One or more steps or operations of routine 600 may be embodied as one or more operations of system 100 as shown in FIG. 1 and/or system 200 as shown in FIG. 2. The operations in routine 600 may be performed in the order presented, in a different order, or simultaneously. Further, in some exemplary embodiments, some of the operations may be omitted, added, modified, skipped, or the like without departing from the scope of the invention.

In accordance with certain embodiments, routine 600 comprises a participant onboarding workflow for remote clinical trial management configured to integrate secure identity verification and informed consent procedures. Routine 600 commences when a deep-link token is sent from server onboarding modules 652 to the participant client 654 (step 602). Upon receiving this token, participant client 654 launches an onboarding web-view interface by opening the provided link (step 604). In the subsequent steps, participant client 654 captures an identification image along with a selfie of the participant (step 606), and concurrently, authenticates device possession via two-factor authentication (step 608). Following these initial verifications, the onboarding system renders the informed consent workflow, presenting the participant with necessary trial information and consent forms (step 610). The participant then completes the informed consent workflow and undergoes a knowledge check to verify understanding (step 612).

These captured identification images and selfies are transmitted to an external Know Your Customer (KYC) service 656 for validation (step 614). Upon successful validation, a token indicating approval is returned to confirm the participant's identity. Concurrently, successful completion of the informed consent workflow and knowledge check is verified by the server onboarding modules (step 618). Once the participant's identity and comprehension of the consent information have been confirmed, the participant captures either an electronic and/or voice signature to formalize their consent to participate in the clinical trial (step 620). The resulting onboarding data, including consent forms and verification data, is securely stored in an electronic Informed Consent Form (eICF) repository (step 622). Finally, the system updates an audit ledger to record the onboarding event to ensure traceability and compliance (step 624).

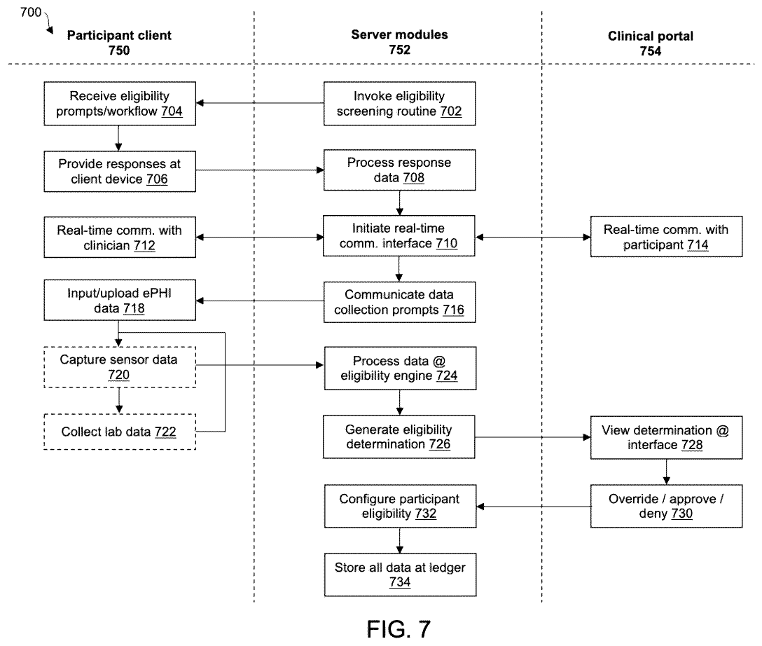

Referring now to FIG. 7, a logic flow diagram of a routine 700 of the system for automated remote management of clinical trials is shown. In accordance with certain aspects of the present disclosure, the conversational system for automated remote management of clinical trials may comprise system 100 as shown and described in FIG. 1. One or more steps or operations of routine 700 may be embodied as one or more operations of system 100 as shown in FIG. 1 and/or system 200 as shown in FIG. 2. The operations in routine 700 may be performed in the order presented, in a different order, or simultaneously. Further, in some exemplary embodiments, some of the operations may be omitted, added, modified, skipped, or the like without departing from the scope of the invention.

In accordance with certain aspects of the present disclosure, routine 700 comprises a comprehensive participant eligibility determination workflow for remote clinical trials. In accordance with certain embodiments, routine 700 comprises a plurality of coordinated operations between participant client devices 750, server modules 752, and a clinical portal 754. In accordance with certain embodiments, routine 700 may be initiated via one or more steps or operations at server modules 752 to invoke an eligibility screening routine (step 702). Eligibility prompts and workflow instructions are subsequently delivered to participant client device 750 (step 704). Eligibility prompts and workflow instructions may be delivered via one or more modalities, including a web-based workflow, a conversational chat interface, and/or a voice-based interface. A participant user interacts with participant client device 750 to provide necessary responses for eligibility assessment (step 706). In accordance with certain embodiments, eligibility information may include, for example, demographics, medical history, symptom data, concomitant medications, and the like. Server modules 752 then process the participant-provided response data (step 708). In accordance with certain embodiments, routine 700 may comprise one or more steps or operations to initiate real-time communication interface (step 710) between a participant user (step 712) and a clinician user (step 714). The real-time communication interface may be established for the purpose of gathering or verifying one or more clinical eligibility criteria. Throughout the interaction, server modules 752 communicate specific data collection prompts to the participant (step 716). The participant responds by inputting or uploading electronic Protected Health Information (ePHI) data (step 718) at participant client 750. Additionally, relevant sensor data may be captured by one or more connected devices (step 720), such as a wearable activity tracker, thermometer, blood pressure monitor, pulse oximeter, heart rate monitor and the like. In certain embodiments, routine 700 may comprise one or more steps or operations for collecting laboratory data, such as scheduling a lab test for the participant and/or receiving a data feed from a connected LIMS system (step 722).

In accordance with certain aspects of the present disclosure, routine 700 may comprise one or more steps or operations for receiving the eligibility data (e.g., participant-reported data, device/sensor data, lab data) at server module 752 and processing the data via a specialized eligibility engine (step 724). The eligibility engine may be configured to exercise a logic flow that is configured according to a trial eligibility protocol, resulting in the generation of an eligibility determination for the participant (step 726). Routine 700 may comprise one or more steps or operations for displaying the eligibility determination to the clinician via a dedicated interface at clinical portal 754 (step 728). Clinicians have the capability to review, override, approve, or deny the determination as deemed appropriate (e.g., in accordance with the clinical trial eligibility protocol) (step 730). Based on clinician decisions, routine 700 comprises one or more steps or operations for configuring the participant's eligibility status for the clinical trial (step 732). In accordance with certain aspects of the present disclosure, comprehensive data related to the participant's responses (including sensor data and lab data), clinician interactions, and the final eligibility determination are securely stored in an audit ledger to ensure transparency and compliance (step 734).

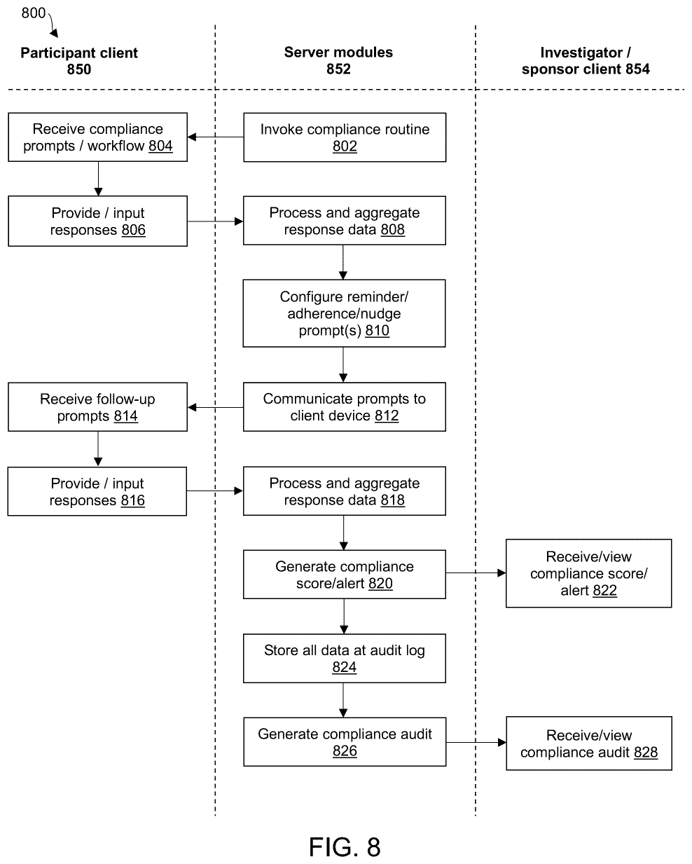

Referring now to FIG. 8, a logic flow diagram of a routine 800 of the system for automated remote management of clinical trials is shown. In accordance with certain aspects of the present disclosure, the conversational system for automated remote management of clinical trials may comprise system 100 as shown and described in FIG. 1. One or more steps or operations of routine 800 may be embodied as one or more operations of system 100 as shown in FIG. 1 and/or system 200 as shown in FIG. 2. The operations in routine 800 may be performed in the order presented, in a different order, or simultaneously. Further, in some exemplary embodiments, some of the operations may be omitted, added, modified, skipped, or the like without departing from the scope of the invention.

In accordance with certain aspects of the present disclosure, routine 800 comprises a participant-compliance monitoring and adherence workflow that operates within a remote-trial architecture (e.g., system 100 of FIG. 1). In accordance with certain embodiments, routine 800 comprises one or more coordinating coordinated operations among a participant client device 850, cloud-hosted server modules 852, and an investigator/sponsor client 854. In accordance with certain embodiments, routine 800 begins when server modules 852 invoke a compliance routine (step 802). Immediately thereafter, server modules 852 may perform one or more steps or operations for transmitting compliance prompts and an associated workflow to participant client 850 (step 804). The compliance prompts and an associated workflow may be delivered via one or more modalities, including voice-based, a web-based graphical user interface, an app-based graphical user interface, and/or a conversational chat interface. The participant reviews the material and supplies the requested responses (e.g., such as medication-intake confirmations, dosing timestamps, or survey answers) via the client device interface (step 806).

In accordance with certain embodiments, routine 800 proceeds by executing one or more steps or operations (e.g., via server module 852) to process and aggregate the initial response data (step 808) and based upon predefined protocol logic, automatically configure one or more reminder, adherence, or nudge prompts (step 810). The prompts (e.g., push notifications, SMS messages, or in-app alerts, voice-based prompts, conversation text-based prompts) are communicated back to participant client 850 (step 812). Upon receipt, the participant encounters follow-up prompts (step 814) and again provides responses confirming the requested adherence activity (step 816). Response data is processed and aggregated by server modules 852 (step 818), whereupon a compliance engine computes a dynamic compliance score and/or generates an alert indicating non-adherence (step 820) for the participant. The calculated compliance score or alert is transmitted to, and made viewable within, a graphical user interface of investigator/sponsor client 854 (step 822). To ensure immutable traceability, all input data, system-generated prompts, and resulting compliance determinations are persistently stored in a tamper-evident audit log (step 824). In accordance with certain aspects of the present disclosure, routine 800 may comprise one or more steps or operations for compiling audit-log entries into a comprehensive compliance audit (step 826) (e.g., at user-defined intervals or upon request). The resulting audit report, which may include timestamped participant interactions, adherence metrics, and alert histories, is then made available for review by investigators or sponsors through the user interface at investigator/sponsor client 854 (step 828).

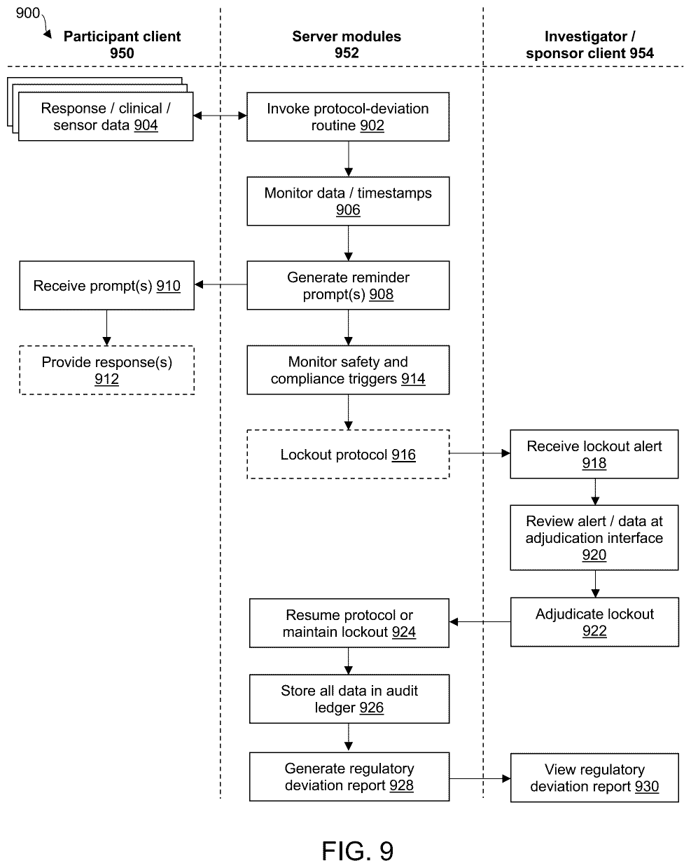

Referring now to FIG. 9, a logic flow diagram of a routine of the system for automated remote management of clinical trials is shown. In accordance with certain aspects of the present disclosure, the conversational system for automated remote management of clinical trials may comprise system 100 as shown and described in FIG. 1. One or more steps or operations of routine 900 may be embodied as one or more operations of system 100 as shown in FIG. 1 and/or system 200 as shown in FIG. 2. The operations in routine 900 may be performed in the order presented, in a different order, or simultaneously. Further, in some exemplary embodiments, some of the operations may be omitted, added, modified, skipped, or the like without departing from the scope of the invention.

In accordance with certain aspects of the present disclosure, routine 900 comprises one or more steps or operations for a protocol-deviation detection and lockout adjudication workflow for remote clinical trials. In accordance with certain embodiments, routine 900 comprises one or more orchestrated interactions among a participant client device 950, cloud-based server modules 952, and an investigator/sponsor client 954. In accordance with certain embodiments, routine 900 is initiated when server modules 952 invoke a protocol-deviation monitoring routine (step 902).

Incoming participant-response data, objective clinical data, and sensor data streams from participant client 950 are ingested (step 904). Routine 900 may comprise one or more steps or operations for time-stamped analytics for continuously monitoring the received data for schedule adherence and threshold compliance (step 906). When deviation heuristics (e.g., reflex logic) indicate a potential lapse, such as a missed dose window, out-of-range vital sign, or overdue survey, routine 900 may comprise one or more steps or operations to automatically generate one or more reminder prompts (step 908). The one or more reminder prompts may be delivered to participant client 950, where they are received (step 910) and answered by the participant (step 912). In accordance with certain embodiments, the one or more reminder prompts may be configured via one or more modalities (e.g., voice-based, text-based, SMS, email, and the like) Routine 900 may comprise one or more steps or operations to evaluate the responses while continuing to monitor both safety-related and protocol-compliance triggers in real time (step 914).

If the deviation persists beyond configurable limits, or if a critical safety trigger is detected, routine 900 may comprise one or more steps or operations to enforce a lockout protocol (step 916) to temporarily suspend further participant activity (e.g., dosing, data entry) to mitigate elevated risk. In certain embodiments, a lockout alert may be simultaneously transmitted to investigator/sponsor client 954 (step 918). Within an investigator/sponsor interface, an adjudication dashboard may present the alert together with the underlying data trace (step 920). The investigator user may review the information and makes an adjudication decision (step 922), selecting either to resume the participant's protocol engagement or to maintain the lockout pending additional investigation (step 924). In accordance with certain aspects of the present disclosure, all events, including raw data, prompts, participant responses, and adjudication outcomes, are immutably written to an audit ledger (step 926). Routine 900 may comprise one or more steps or operations for compiling a detailed regulatory deviation report to ensure compliance with regulatory obligations (step 928). In accordance with certain embodiments, the investigator or sponsor can access and review the detailed regulatory deviation report through the investigator/sponsor interface (step 930). In accordance with certain aspects of the present disclosure, routine 900 ensures rapid detection of protocol deviations, enforces participant safety through automated lockouts, and provides auditable, regulator-ready documentation of all deviation-related activities.

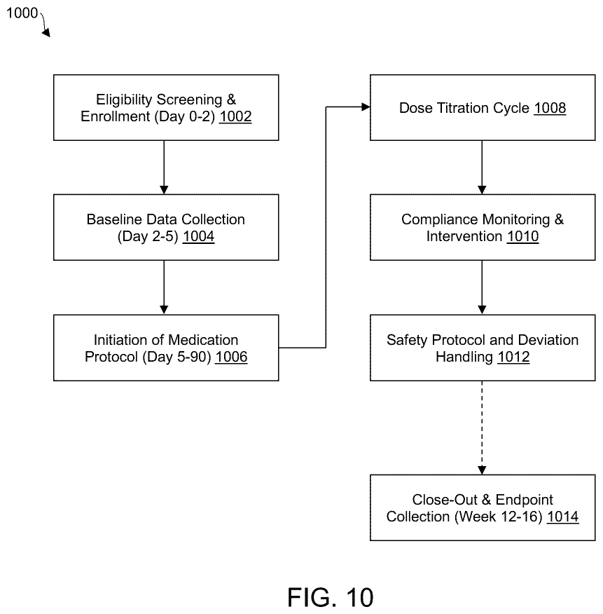

FIG. 10 illustrates a process flow 1000 for an exemplary case study for a cardiovascular clinical trial involving home-based titration of guideline-directed medical therapy (GDMT) for heart failure patients, structured according to the three-environment architecture introduced in FIG. 1. In accordance with certain aspects of the present disclosure, process flow 1000 begins at step 1002 with Eligibility Screening and Enrollment, during which referred patients interact with a conversational AI system operating within the participant environment. In accordance with certain embodiments, the system gathers screening data through a structured voice dialogue, while backend integration with EHR sources provides relevant clinical values, including left ventricular ejection fraction (LVEF), serum creatinine, and NT-proBNP. The eligibility engine applies protocol-defined rules to this multi-modal data to generate a real-time eligibility determination.

Simultaneously, clinicians can review flagged edge cases within the investigator/sponsor dashboard and override automated eligibility decisions when warranted. Upon approval, participants complete a Part 11-compliant eConsent process using an in-app guided workflow, and the completed consent form is hashed and entered into the immutable audit ledger maintained within the application-computing environment.

In accordance with certain embodiments, process flow 1000 proceeds to step 1004, Baseline Data Collection, in which participants begin streaming physiologic data through RPM devices including connected blood pressure monitors, digital weight scales, and wearable heart rate sensors. Additional laboratory parameters such as creatinine, potassium, and NT-proBNP are pulled from integrated EHR or lab sources and reconciled against expected visit windows. All incoming data is time-aligned and stored as the patient's pre-intervention baseline, forming the reference dataset for future titration decisions. At step 1006, Initiation of Medication Protocol, participants begin the prescribed heart failure therapy on or around study day 5. A conversational module provides guided instruction for first-dose administration and immediate safety checks, confirming vitals and adherence post-dose. The system automatically schedules the first titration checkpoint and initializes a dose-titration schedule based on protocol-defined cadence, typically every 14 days.

In accordance with certain aspects of the present disclosure, the core of the iterative treatment process is captured in step 1008, Dose Titration Cycle, where the system evaluates whether criteria for dose advancement, maintenance, or holding are met. During each cycle, the system ingests and analyzes a predefined panel of clinical indicators, including blood pressure, heart rate, weight trends, and self-reported symptoms. The algorithmic logic engine compares these inputs against titration thresholds, dynamically adjusts the visit schedule, and generates new prompts or alerts as needed. If the participant meets criteria for dose escalation, the system provides instruction and records confirmation of dose change; if safety criteria are not met, the system may defer titration or activate lockout protocols. Parallel to titration, step 1010, Compliance Monitoring and Intervention, continuously monitors participant adherence using data from device synchronization logs, missed check-ins, and biometric trends. When early signs of disengagement or noncompliance emerge, such as a delayed weight transmission or inconsistent blood pressure uploads, the system deploys tiered interventions, ranging from automated nudges to escalation for staff follow-up. The adherence engine also incorporates sentiment and stress markers derived from voice input, flagging deteriorations in psychological state that could impact compliance or safety. Step 1012, Safety Protocol and Deviation Handling, governs lockout criteria and safety thresholds. If the participant's vitals cross protocol-defined boundaries (e.g., systolic blood pressure below 90 mmHg, heart rate below 50 bpm, significant weight gain, or lab anomalies), the system automatically pauses titration, drafts a deviation log entry, and pushes an alert to the sponsor dashboard. The investigator may then adjudicate the safety event, resume protocol, or withdraw the participant, all actions that are documented and linked via cryptographic hashes to the audit trail.

Following the titration phase, step 1014, Close-Out and Endpoint Collection, initiates a structured offboarding sequence. The system issues final conversational assessments, including validated quality-of-life instruments such as the Kansas City Cardiomyopathy Questionnaire (KCCQ), and confirms medication compliance via inventory review and self-reported usage. The system aggregates all endpoint data (e.g., ranging from vitals and lab values to patient-reported outcomes) and compiles a secure, submission-ready dossier suitable for regulatory review. A close-out report is auto-generated, and the full participant record is sealed in the audit ledger, completing the end-to-end evidence trail.

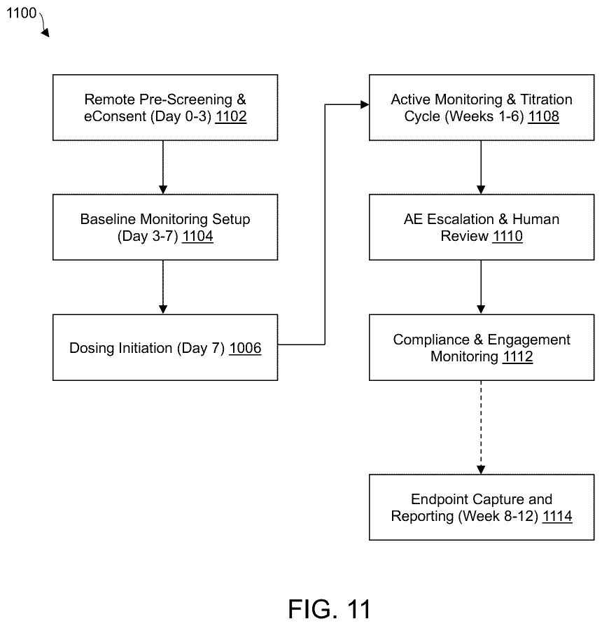

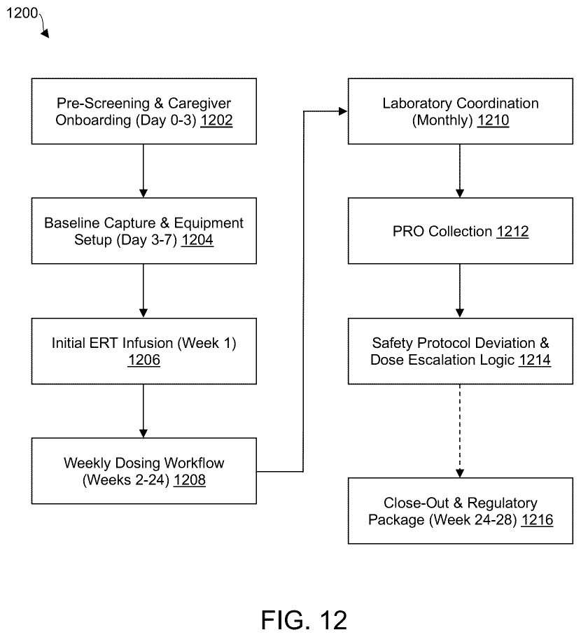

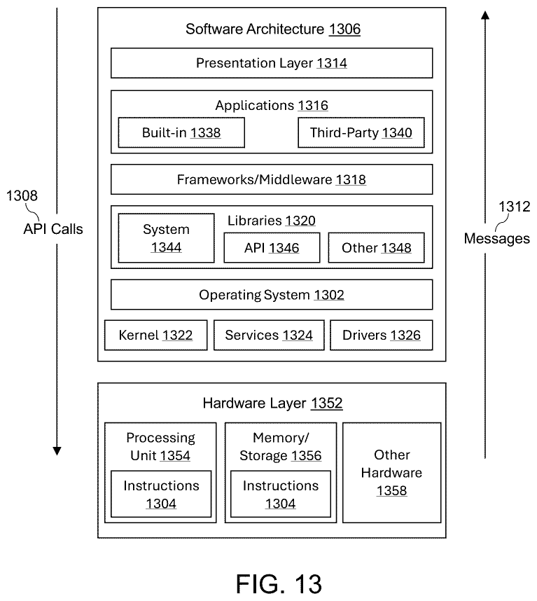

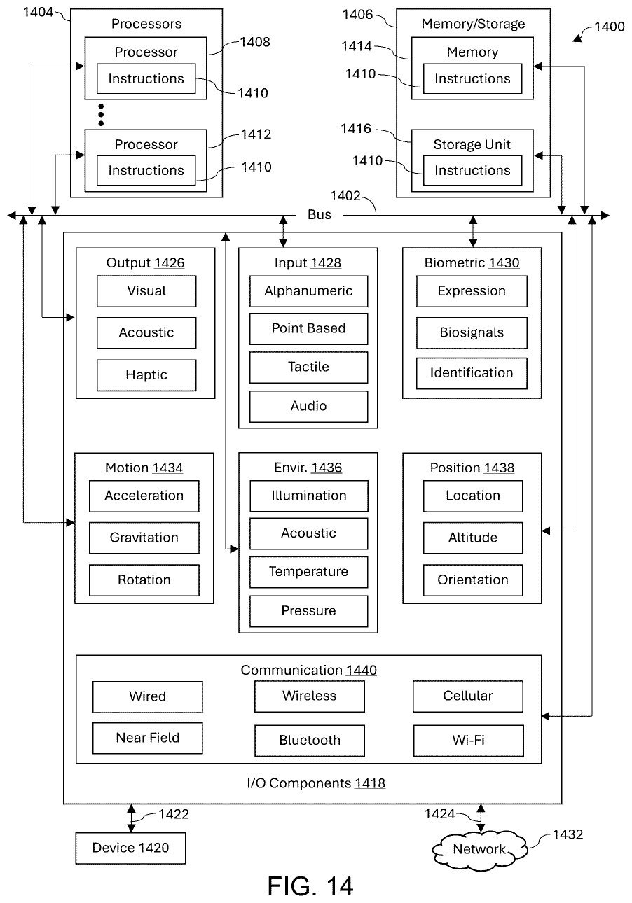

Referring now to FIG. 11, an end-to-end case-study flow 1100 for a fully decentralized oncology trial is shown. In accordance with certain aspects of the present disclosure, the fully decentralized oncology trial is configured wherein patients with advanced solid tumors self-titrate an investigational oral kinase inhibitor while the platform introduced in FIG. 1 manages safety, adherence, and data integrity. In accordance with certain aspects of the present disclosure, flow 1100 is anchored in the same three-environment architecture as described herein, thereby showing how voice-centric AI, remote-patient-monitoring devices, and clinician dashboards cooperate to deliver a home-based regimen that satisfies 21 CFR Part 11 record-keeping requirements. In accordance with certain aspects of the present disclosure, flow 1100 begins at step 1102, Remote Pre-Screening and eConsent (e.g., days 0-3 in the clinical trial workflow). Within the participant environment, the conversational agent conducts a voice interview covering screening questions including, but not limited to, tumor type, prior lines of therapy, and Eastern Cooperative Oncology Group (ECOG) performance status. Simultaneously, the system calls electronic-health-record (EHR) APIs to retrieve structured laboratory values (e.g., complete blood count, liver-function tests, renal panel) and applies optical-character-recognition plus natural-language understanding to parse any scanned imaging or pathology reports. The eligibility engine fuses these multimodal inputs against protocol criteria and produces a real-time inclusion verdict that investigators may override from the sponsor dashboard. Once eligible, the patient completes an in-app, voice-supported informed-consent sequence; the signed form and an audio assent clip are time-stamped and hashed into the tamper-evident audit ledger.

At step 1104, Baseline Monitoring Setup (e.g., scheduled for days 3-7 in the clinical trial workflow), the participant receives a device kit that may include a smart pill bottle or blister-pack sensor, a digital thermometer, a connected blood-pressure cuff, and an optional wearable patch for heart-rate and QT-interval monitoring. Conversational training scripts teach the user how to pair each device, describe adverse-event (AE) reporting etiquette, and administer baseline questionnaires such as PHQ-2 for mood and a confirmatory ECOG assessment. All baseline vitals and survey responses are uploaded through the participant client to the application-computing environment, where they establish the reference values for subsequent titration analytics. At step 1106, Dosing Initiation (e.g., occurring on day 7 in the clinical trial workflow). The system prompts the participant to ingest the starting daily dose (XX mg) and verifies adherence through both pill-sensor telemetry and a brief voice confirmation. The AI then reiterates dietary restrictions and symptom-tracking expectations and configures a reminder that triggers if the pill sensor fails to register an opening within the permitted window.

In accordance with certain aspects of the present disclosure, at step 1108, Active Monitoring and Titration Cycle (e.g., weeks 1-6 in the clinical trial workflow), the platform maintains a two-tier conversational cadence: a daily screener captures gastrointestinal symptoms such as nausea, vomiting, or diarrhea, while a weekly structured interview queries fatigue, neuropathy, dermatologic changes, mucositis, and fever. A toxicity-detection algorithm continuously analyses these self-reports alongside biometric feeds (e.g., temperature logs for pyrexia, blood-pressure and heart-rate series for hemodynamic instability) and maps emergent terms to MedDRA categories with confidence scores. If laboratory or symptomatic thresholds are met, adaptive protocol logic auto-generates one of several actions: maintain dose, reduce dose, hold therapy, or discontinue treatment entirely, all while documenting the rationale and lining up the next check-in schedule.

When an AE crosses predefined severity or novelty limits, flow 1100 proceeds to step 1110, AE Escalation and Human Review. In accordance with certain embodiments, the platform converts the triggering voice snippet and supporting vitals into a timestamped AE record, pushes an alert with playback controls to the investigator dashboard, and suspends further titration. A clinician can then authorize continuation, mandate a hold, or withdraw the participant; the system resumes or adapts the study flow immediately upon adjudication, logging each decision in the immutable ledger. In accordance with certain embodiments, step 1112, Parallel Compliance and Engagement Monitoring, tracks behavioral metrics such as missed screeners, delayed vitals uploads, or inconsistencies (e.g., fever recorded without fatigue report). An engagement-scoring model grades each participant; declining scores escalate the case to a study coordinator, prompting personalized outreach designed to restore adherence before protocol deviation thresholds are breached.

In accordance with certain embodiments, step 1114, Endpoint Capture and Reporting, covers weeks 8-12 in the clinical trial workflow. At step 1114, a voice-first administration of validated patient-reported outcome instruments, including the EORTC QLQ-C30 and FACIT-Fatigue, collects quality-of-life data, while the system verifies AE resolution timelines and final medication exposure. An audit-quality dossier compiles medication-adherence graphs, dose-hold histories, AE logs, escalation trails, and adjudication notes. The platform then autogenerates structured AE narratives and protocol-deviation files in Part 11-compliant format, ready for direct submission to regulatory authorities.

Referring now to FIG. 12, a process flow diagram of an end-to-end case-study flow 1200 for a rare-disease trial is shown. In accordance with certain aspects of the present disclosure, the rare-disease trial may comprise a clinical trial in which children with genetically confirmed Pompe disease receive enzyme-replacement therapy (ERT) entirely at home, yet every action, decision, and datapoint is orchestrated by the conversational architecture introduced in FIG. 1.

In accordance with certain aspects of the present disclosure, at step 1202, Pre-Screening and Caregiver Onboarding (e.g., day 0-3 in the clinical trial workflow), a conversational AI engages the child's caregiver by smartphone or smart speaker, collects disease-history items such as age at motor-symptom onset, ventilator use, and GAA genotype, and prompts secure upload of diagnostic artifacts (dried-blood-spot cards, biopsy reports). In certain embodiments, a know-your-customer pipeline verifies caregiver identity through a selfie-plus-ID check, after which the caregiver completes a mobile eConsent while the child provides an age-appropriate audio assent; the signed forms are hashed into the immutable audit ledger. At step 1204, Baseline Capture and Equipment Setup (e.g., day 3-7 in the clinical trial workflow), the participant is equipped with a BLUETOOTH spirometer, digital scale, pulse oximeter, and (optionally) an EMG patch (e.g., where ventilation or tone monitoring is relevant). Voice-guided calibration ensures the child produces best-effort spirometry, and all baseline vitals stream into the application-computing environment to establish reference values for future dose logic.

In accordance with certain aspects of the present disclosure, a first supervised infusion occurs at step 1206, Initial ERT Infusion (e.g. at week 1 in the clinical trial workflow), either during a clinic visit or via an in-home nurse. Baseline laboratory panels are drawn, and an AI training module delivers multimedia instructions on infusion technique and adverse-event (AE) response, confirming comprehension with a repeat-back quiz before releasing the nurse. From weeks to 24, the study cycles through step 1208, Weekly Dosing Workflow. In accordance with certain embodiments, twenty-four hours before each infusion, the system conducts a conversational symptom screen (e.g., “Has [child's name] had a fever in the past 24 hours?”) and verifies supply availability and site preparation. During the infusion, connected sensors passively capture heart-rate, oxygen saturation, and motion data; at 24- and 72-hour marks the AI checks for rash, vomiting, apnea, or breathing changes, chaining every prompt, response, and biometric packet to the audit ledger.

At step 1210, a monthly Laboratory Coordination routine reminds caregivers 48 hours before at-home sample collection, guide them through voice or video instructions (e.g., “Place the dried-blood-spot card on a clean surface . . . ”) and track shipment receipt. Returned results (e.g., ALT, AST, antibody titers) are fed directly into protocol-continuation logic. At step 1212, a Patient-Reported-Outcome (PRO) Collection deploys voice-driven milestone checklists (e.g., “Can your child climb stairs unaided?”), PROMIS-Caregiver-Fatigue surveys, and pediatric quality-of-life instruments. Missed assessments or vocal-stress cues trigger graded nudges or live coordinator outreach, keeping engagement high.

In accordance with certain aspects of the present disclosure, safety and dose progression converge at step 1214, Safety-Protocol Deviation and Dose-Escalation Logic. In accordance with certain embodiments, adaptive rules permit an ERT increase only after four consecutive AE-free infusions with stable labs; the system enforces an automatic lockout for any Grade 2+AE, missed laboratory, or newly reported symptom. When a lockout fires, the platform compiles a flagged report with full audit context; an investigator may release or uphold the lock via an encrypted dashboard, and the decision itself is cryptographically chained to the child's record. In accordance with certain embodiments, at step 1216, Close-Out and Regulatory Package (e.g., weeks 24-28 in the clinical trial workflow) administers final milestone and PRO assessments, reconciles the last lab values, and auto-generates a submission-ready dossier that includes a complete timeline of caregiver interactions, adherence traces, dose holds, and time-stamped AE logs formatted as CDISC- and HL7-compliant files. The audit trail is sealed, providing regulators with an unbroken line of sight from onboarding through study completion.

FIG. 13 is a block diagram illustrating an exemplary software architecture 1306, which may be used in conjunction with various hardware architectures herein described. FIG. 13 is a non-limiting example of a software architecture, and it will be appreciated that many other architectures may be implemented to facilitate the functionality described herein. The software architecture 1306 may execute on hardware such as machine 1400 of FIG. 14 that includes, among other things, processors 1404, memory 1414, and I/O components 1418. A representative hardware layer 1352 is illustrated and can represent, for example, the machine 1400 of FIG. 14. The representative hardware layer 1352 includes a processing unit 1354 having associated executable instructions 1304. Executable instructions 1304 represent the executable instructions of the software architecture 1306, including implementation of the methods, components and so forth described herein. The hardware layer 1352 also includes memory or storage modules memory/storage 1356, which also have executable instructions 1304. The hardware layer 1352 may also comprise other hardware 1358.

As used herein, the term “component” may refer to a device, physical entity or logic having boundaries defined by function or subroutine calls, branch points, application program interfaces (APIs), or other technologies that provide for the partitioning or modularization of particular processing or control functions. Components may be combined via their interfaces with other components to carry out a machine process. A component may be a packaged functional hardware unit designed for use with other components and a part of a program that usually performs a particular function of related functions.

Components may constitute either software components (e.g., code embodied on a machine-readable medium) or hardware components. A “hardware component” is a tangible unit capable of performing certain operations and may be configured or arranged in a certain physical manner. In various exemplary embodiments, one or more computer systems (e.g., a standalone computer system, a client computer system, or a server computer system) or one or more hardware components of a computer system (e.g., a processor or a group of processors) may be configured by software (e.g., an application or application portion) as a hardware component that operates to perform certain operations as described herein. A hardware component may also be implemented mechanically, electronically, or any suitable combination thereof. For example, a hardware component may include dedicated circuitry or logic that is permanently configured to perform certain operations.

A hardware component may be a special-purpose processor, such as a Field-Programmable Gate Array (FPGA) or an Application Specific Integrated Circuit (ASIC). A hardware component may also include programmable logic or circuitry that is temporarily configured by software to perform certain operations. For example, a hardware component may include software executed by a processor or other programmable processor. Once configured by such software, hardware components become specific machines (or specific components of a machine) uniquely tailored to perform the configured functions and are no longer processors. It will be appreciated that the decision to implement a hardware component mechanically, in dedicated and permanently configured circuitry, or in temporarily configured circuitry (e.g., configured by software) may be driven by cost and time considerations.

A processor may be, or in include, any circuit, circuitry, or virtual circuit (a physical circuit emulated by logic executing on an actual processor) that manipulates data values according to control signals (e.g., “commands”, “op codes”, “machine code”, etc.) and which produces corresponding output signals that are applied to operate a machine. A processor may, for example, be a Central Processing Unit (CPU), a Reduced Instruction Set Computing (RISC) processor, a Complex Instruction Set Computing (CISC) processor, a Graphics Processing Unit (GPU), a Digital Signal Processor (DSP), an Application Specific Integrated Circuit (ASIC), a Radio-Frequency Integrated Circuit (RFIC) or any combination thereof. A processor may further be a multi-core processor having two or more independent processors (sometimes referred to as “cores”) that may execute instructions contemporaneously. The processor as used herein may be a hardware component, which is in at least one of the devices, systems, servers and the like. The processor may include multiple cores and may be spread across multiple devices. The processor includes circuitry to execute instructions relating to the methods and structures described herein for determining relationships and outputting relationship data that is used by various device and their users.

Accordingly, the phrase “hardware component” (or “hardware-implemented component”) should be understood to encompass a tangible entity, be that an entity that is physically constructed, permanently configured (e.g., hardwired), or temporarily configured (e.g., programmed) to operate in a certain manner or to perform certain operations described herein.

Considering embodiments in which hardware components are temporarily configured (e.g., programmed), each of the hardware components need not be configured or instantiated at any one instance in time. For example, where a hardware component comprises a processor configured by software to become a special-purpose processor, the processor may be configured as respectively different special-purpose processors (e.g., comprising different hardware components) at different times. Software accordingly configures a particular processor or processors, for example, to constitute a particular hardware component at one instance of time and to constitute a different hardware component at a different instance of time. Hardware components can provide information to, and receive information from, other hardware components. Accordingly, the described hardware components may be regarded as being communicatively coupled. Where multiple hardware components exist contemporaneously, communications may be achieved through signal transmission (e.g., over appropriate circuits and buses) between or among two or more of the hardware components. In embodiments in which multiple hardware components are configured or instantiated at different times, communications between such hardware components may be achieved, for example, through the storage and retrieval of information in memory structures to which the multiple hardware components have access.

For example, one hardware component may perform an operation and store the output of that operation in a memory device to which it is communicatively coupled. A further hardware component may then, at a later time, access the memory device to retrieve and process the stored output. Hardware components may also initiate communications with input or output devices, and can operate on a resource (e.g., a collection of information). The various operations of example methods described herein may be performed, at least partially, by one or more processors that are temporarily configured (e.g., by software) or permanently configured to perform the relevant operations. Whether temporarily or permanently configured, such processors may constitute processor-implemented components that operate to perform one or more operations or functions described herein. As used herein, “processor-implemented component” refers to a hardware component implemented using one or more processors. Similarly, the methods described herein may be at least partially processor-implemented, with a particular processor or processors being an example of hardware. For example, at least some of the operations of a method may be performed by one or more processors or processor-implemented components.

Moreover, the one or more processors may also operate to support performance of the relevant operations in a “cloud computing” environment or as a “software as a service” (SaaS). For example, at least some of the operations may be performed by a group of computers (as examples of machines including processors), with these operations being accessible via a network (e.g., the Internet) and via one or more appropriate interfaces (e.g., an Application Program Interface (API)). The performance of certain of the operations may be distributed among the processors, not only residing within a single machine, but deployed across a number of machines. In some exemplary embodiments, the processors or processor-implemented components may be located in a single geographic location (e.g., within a home environment, an office environment, or a server farm). In other exemplary embodiments, the processors or processor-implemented components may be distributed across a number of geographic locations.

In the exemplary architecture of FIG. 13, the software architecture 1306 may be conceptualized as a stack of layers where each layer provides particular functionality. For example, the software architecture 1306 may include layers such as an operating system 1302, libraries 1320, applications 1316 and a presentation layer 1314. Operationally, the applications 1316 or other components within the layers may invoke application programming interface (API) API calls 1308 through the software stack and receive messages 1312 in response to the API calls 1308. The layers illustrated are representative in nature and not all software architectures have all layers. For example, some mobile or special purpose operating systems may not provide a frameworks/middleware 1318, while others may provide such a layer. Other software architectures may include additional or different layers.

The operating system 1302 may manage hardware resources and provide common services. The operating system 1302 may include, for example, a kernel 1322, services 1324 and drivers 1326. The kernel 1322 may act as an abstraction layer between the hardware and the other software layers. For example, the kernel 1322 may be responsible for memory management, processor management (e.g., scheduling), component management, networking, security settings, and so on. The services 1324 may provide other common services for the other software layers. The drivers 1326 are responsible for controlling or interfacing with the underlying hardware. For instance, the drivers 1326 include display drivers, camera drivers, Bluetooth® drivers, flash memory drivers, serial communication drivers (e.g., Universal Serial Bus (USB) drivers), Wi-Fi® drivers, audio drivers, power management drivers, and so forth depending on the hardware configuration.

The libraries 1320 provide a common infrastructure that is used by the applications 1316 or other components or layers. The libraries 1320 provide functionality that allows other software components to perform tasks in an easier fashion than to interface directly with the underlying operating system 1302 functionality (e.g., kernel 1322, services 1324 or drivers 1326). The libraries 1320 may include system libraries 1344 (e.g., C standard library) that may provide functions such as memory allocation functions, string manipulation functions, mathematical functions, and the like. In addition, the libraries 1320 may include API libraries 1346 such as media libraries (e.g., libraries to support presentation and manipulation of various media format such as MPREG4, H.264, MP3, AAC, AMR, JPG, PNG), graphics libraries (e.g., an OpenGL framework that may be used to render 2D and 3D in a graphic content on a display), database libraries (e.g., SQLite that may provide various relational database functions), web libraries (e.g., WebKit that may provide web browsing functionality), and the like. The libraries 1320 may also include a wide variety of other libraries 1348 to provide many other APIs to the applications 1316 and other software components/modules.

The frameworks/middleware 1318 (also sometimes referred to as middleware) provide a higher-level common infrastructure that may be used by the applications 1316 or other software components/modules. For example, the frameworks/middleware 1318 may provide various graphic user interface (GUI) functions, high-level resource management, high-level location services, and so forth. The frameworks/middleware 1318 may provide a broad spectrum of other APIs that may be utilized by the applications 1316 or other software components/modules, some of which may be specific to a particular operating system 1302 or platform.

The applications 1316 include built-in applications 1338 or third-party applications 1340. The third-party applications 1340 may invoke the API calls 1308 provided by the operating system 1302 to facilitate functionality described herein.

The applications 1316 may use built in operating system functions (e.g., kernel 1322, services 1324 or drivers 1326), libraries 1320, and frameworks/middleware 1318 to create user interfaces to interact with users of the system. Alternatively, or additionally, in some systems interactions with a user may occur through a presentation layer, such as presentation layer 1314. In these systems, the application/component “logic” can be separated from the aspects of the application/component that interact with a user.

FIG. 14 is a block diagram illustrating components (also referred to herein as “modules”) of a machine 1400, according to some exemplary embodiments, able to read instructions from a machine-readable medium (e.g., a machine-readable storage medium) and perform any one or more of the methodologies discussed herein. Specifically, FIG. 14 shows a diagrammatic representation of the machine 1400 in the example form of a computer system, within which instructions 1410 (e.g., software, a program, an application, an applet, an app, or other executable code) for causing the machine 1400 to perform any one or more of the methodologies discussed herein may be executed. As such, the instructions 1410 may be used to implement modules or components described herein. The instructions 1410 transform the non-programmed machine 1400 into a particular machine 1400 programmed to carry out the described and illustrated functions in the manner described. In alternative embodiments, the machine 1400 operates as a standalone device or may be coupled (e.g., networked) to other machines. In a networked deployment, the machine 1400 may operate in the capacity of a server machine or a client machine in a server-client network environment, or as a peer machine in a peer-to-peer (or distributed) network environment. The machine 1400 may comprise, but not be limited to, a server computer, a client computer, a personal computer (PC), a laptop computer, a network router, a network switch, a network bridge, or any machine capable of executing the instructions 1410, sequentially or otherwise, that specify actions to be taken by machine 1400. Further, while only a single machine 1400 is illustrated, the term “machine” shall also be taken to include a collection of machines that individually or jointly execute the instructions 1410 to perform any one or more of the methodologies discussed herein.

The machine 1400 may include processors 1404, memory memory/storage 1406, and I/O components 1418, which may be configured to communicate with each other such as via a bus 1402. The memory/storage 1406 may include a memory 1414, such as a main memory, or other memory storage, and a storage unit 1416, both accessible to the processors 1404 such as via the bus 1402. The storage unit 1416 and memory 1414 store the instructions 1410 embodying any one or more of the methodologies or functions described herein. The instructions 1410 may also reside, completely or partially, within the memory 1414, within the storage unit 1416, within at least one of the processors 1404 (e.g., within the processor's cache memory), or any suitable combination thereof, during execution thereof by the machine 1400. Accordingly, the memory 1414, the storage unit 1416, and the memory of processors 1404 are examples of machine-readable media.

As used herein, the term “machine-readable medium,” “computer-readable medium,” or the like may refer to any component, device or other tangible media able to store instructions and data temporarily or permanently. Examples of such media may include, but is not limited to, random-access memory (RAM), read-only memory (ROM), buffer memory, flash memory, optical media, magnetic media, cache memory, other types of storage (e.g., Erasable Programmable Read-Only Memory (EEPROM)) or any suitable combination thereof. The term “machine-readable medium” should be taken to include a single medium or multiple media (e.g., a centralized or distributed database, or associated caches and servers) able to store instructions. The term “machine-readable medium” may also be taken to include any medium, or combination of multiple media, that is capable of storing instructions (e.g., code) for execution by a machine, such that the instructions, when executed by one or more processors of the machine, cause the machine to perform any one or more of the methodologies described herein. Accordingly, a “machine-readable medium” may refer to a single storage apparatus or device, as well as “cloud-based” storage systems or storage networks that include multiple storage apparatus or devices. The term “machine-readable medium” excludes signals per se.

The I/O components 1418 may include a wide variety of components to provide a user interface for receiving input, providing output, producing output, transmitting information, exchanging information, capturing measurements, and so on. The specific I/O components 1418 that are included in the user interface of a particular machine 1400 will depend on the type of machine. It will be appreciated that the I/O components 1418 may include many other components that are not shown in FIG. 14. The I/O components 1418 are grouped according to functionality merely for simplifying the following discussion and the grouping is in no way limiting. In various exemplary embodiments, the I/O components 1418 may include output components 1426 and input components 1428. The output components 1426 may include visual components (e.g., a display such as a plasma display panel (PDP), a light emitting diode (LED) display, a liquid crystal display (LCD), a projector, or a cathode ray tube (CRT)), acoustic components (e.g., speakers), other signal generators, and so forth. The input components 1428 may include alphanumeric input components (e.g., a keyboard, a touch screen configured to receive alphanumeric input, a photo-optical keyboard, or other alphanumeric input components), point based input components (e.g., a mouse, a touchpad, a trackball, a joystick, a motion sensor, or other pointing instrument), tactile input components (e.g., a physical button, a touch screen that provides location or force of touches or touch gestures, or other tactile input components), audio input components (e.g., a microphone), and the like. The input components 1428 may also include one or more image-capturing devices, such as a digital camera for generating digital images or video.

In further exemplary embodiments, the I/O components 1418 may include biometric components 1430, motion components 1434, environmental environment components 1436, or position components 1438, as well as a wide array of other components. One or more of such components (or portions thereof) may collectively be referred to herein as a “sensor component” or “sensor” for collecting various data related to the machine 1400, the environment of the machine 1400, a user of the machine 1400, or a combinations thereof.

Communication may be implemented using a wide variety of technologies. The I/O components 1418 may include communication components 1440 operable to couple the machine 1400 to a network 1432 or devices 1420 via coupling 1422 and coupling 1424 respectively. For example, the communication components 1440 may include a network interface component or other suitable device to interface with the network 1432. In further examples, communication components 1440 may include wired communication components, wireless communication components, cellular communication components, Near Field Communication (NFC) components, Bluetooth® components (e.g., Bluetooth® Low Energy), Wi-Fi® components, and other communication components to provide communication via other modalities. The devices 1420 may be another machine or any of a wide variety of peripheral devices (e.g., a peripheral device coupled via a Universal Serial Bus (USB)). Moreover, the communication components 1440 may detect identifiers or include components operable to detect identifiers.

As will be appreciated by one of skill in the art, the present invention may be embodied as a method (including, for example, a computer-implemented process, a business process, and/or any other process), apparatus (including, for example, a system, machine, device, computer program product, and/or the like), or a combination of the foregoing. Accordingly, embodiments of the present invention may take the form of an entirely hardware embodiment, an entirely software embodiment (including firmware, resident software, micro-code, etc.), or an embodiment combining software and hardware aspects that may generally be referred to herein as a “system.” Furthermore, embodiments of the present invention may take the form of a computer program product on a computer-readable medium having computer-executable program code embodied in the medium.

Embodiments of the present invention are described above with reference to flowchart illustrations and/or block diagrams of methods, apparatus (systems), and computer program products. It will be understood that each block of the flowchart illustrations and/or block diagrams, and/or combinations of blocks in the flowchart illustrations and/or block diagrams, can be implemented by computer-executable program code portions. These computer-executable program code portions may be provided to a processor of a general-purpose computer, special purpose computer, or other programmable data processing apparatus to produce a particular machine, such that the code portions, which execute via the processor of the computer or other programmable data processing apparatus, create mechanisms for implementing the functions/acts specified in the flowchart and/or block diagram block or blocks.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, exemplary methods and materials are now described. All publications mentioned herein are incorporated herein by reference to disclose and describe the methods and/or materials in connection with which the publications are cited.

It must be noted that as used herein and in the appended claims, the singular forms “a”, “an,” and “the” include plural referents unless the context clearly dictates otherwise. Thus, for example, reference to “a stimulus” includes a plurality of such stimuli and reference to “the signal” includes reference to one or more signals and equivalents thereof known to those skilled in the art, and so forth.

Any publications discussed herein are provided solely for their disclosure prior to the filing date of the present application. Nothing herein is to be construed as an admission that the present invention is not entitled to antedate such publication by virtue of prior invention. Further, the dates of publication provided may differ from the actual publication dates which may need to be independently confirmed.

Where a phrase similar to “at least one of A, B, or C,” “at least one of A, B, and C,” “one or more A, B, or C,” or “one or more of A, B, and C” is used, it is intended that the phrase be interpreted to mean that A alone may be present in an embodiment, B alone may be present in an embodiment, C alone may be present in an embodiment, or that any combination of the elements A, B and C may be present in a single embodiment; for example, A and B, A and C, B and C, or A and B and C.

As used herein, the term “or” may be construed in either an inclusive or exclusive sense. Moreover, plural instances may be provided for resources, operations, or structures described herein as a single instance. Additionally, boundaries between various resources, operations, modules, engines, and data stores are somewhat arbitrary, and particular operations are illustrated in a context of specific illustrative configurations. Other allocations of functionality are envisioned and may fall within a scope of various embodiments of the present disclosure. In general, structures and functionality presented as separate resources in the example configurations may be implemented as a combined structure or resource. Similarly, structures and functionality presented as a single resource may be implemented as separate resources.

As the phrase is used herein, a processor may be “configured to” perform a certain function in a variety of ways, including, for example, by having one or more general-purpose circuits perform the function by executing particular computer-executable program code embodied in computer-readable medium, and/or by having one or more application-specific circuits perform the function.

Embodiments of the present invention are described above with reference to flowcharts and/or block diagrams. It will be understood that phases of the processes described herein may be performed in orders different than those illustrated in the flowcharts. In other words, the processes represented by the blocks of a flowchart may, in some embodiments, be in performed in an order other that the order illustrated, may be combined or divided, or may be performed simultaneously. It will also be understood that the blocks of the block diagrams illustrated, in some embodiments, merely conceptual delineations between systems and one or more of the systems illustrated by a block in the block diagrams may be combined or share hardware and/or software with another one or more of the systems illustrated by a block in the block diagrams. Likewise, a device, system, apparatus, and/or the like may be made up of one or more devices, systems, apparatuses, and/or the like. For example, where a processor is illustrated or described herein, the processor may be made up of a plurality of microprocessors or other processing devices which may or may not be coupled to one another. Likewise, where a memory is illustrated or described herein, the memory may be made up of a plurality of memory devices which may or may not be coupled to one another.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

While certain exemplary embodiments have been described and shown in the accompanying drawings, it is to be understood that such embodiments are merely illustrative of, and not restrictive on, the broad invention, and that this invention is not limited to the specific constructions and arrangements shown and described, since various other changes, combinations, omissions, modifications and substitutions, in addition to those set forth in the above paragraphs, are possible. Those skilled in the art will appreciate that various adaptations and modifications of the just described embodiments can be configured without departing from the scope and spirit of the invention. Therefore, it is to be understood that, within the scope of the appended claims, the invention may be practiced other than as specifically described herein.

What is claimed is:

1. A computer-implemented method for clinical trial management, the computer-implemented method comprising:
receiving, by at least one processor via a network interface, protocol parameters that define one or more of a dosing schema, a visit schedule, one or more safety or escalation thresholds, and participant-eligibility criteria for a clinical trial for an investigational medical intervention;
configuring, by the at least one processor, a conversational artificial intelligence (AI) model in accordance with the protocol parameters,
wherein configuring the conversational AI model comprises configuring a set of generative voice prompts mapped to the protocol parameters;
configuring, by the at least one processor, an algorithmic logic engine in accordance with the protocol parameters,
wherein the algorithmic logic engine comprises one or more decision rules for processing participant reported data according to the protocol parameters;
transmitting, to a participant device comprising a microphone and a speaker, a first generative voice prompt via a conversational AI agent executing the conversational AI model;
receiving, via the participant device, audio data in response to the first generative voice prompt;
transcribing the audio data into structured textual content and extracting participant data comprising at least one of symptom information, adherence confirmation, or adverse event descriptors;
evaluating, by the algorithmic logic engine, the participant data in combination with objective clinical data received from a physiological sensor device or an external laboratory data feed against the protocol parameters;
dynamically generating, via the conversational AI agent, a second generative voice prompt in response to evaluating the participant data,
wherein the second generative voice prompt comprises a dosage instruction for the investigational medical intervention or a follow-up generative voice prompt configured to request additional information from the participant;
recording, in an electronic record repository, (i) the protocol parameters, (ii) each generative voice prompt, (iii) the audio data, (iv) the structured textual content, (v) a timestamp, (vi) an audit trail indicator, and (vii) any dosage instruction; and
rendering the secure electronic record repository accessible to at least one authenticated sponsor device or investigator client device via a role-based network connection.

2. The computer-implemented method of claim 1 wherein the dosage instruction is dynamically selected according to an adaptive dose escalation model.

3. The computer-implemented method of claim 2 wherein the adaptive dose escalation model comprises logic executable by the algorithmic logic engine to iteratively update a recommended next dose based on accumulating participant data and objective clinical data relative to at least one protocol-defined safety and/or efficacy threshold.

4. The computer-implemented method of claim 1 wherein evaluating the participant data further comprises classifying adverse event descriptors by automatically mapping each descriptor to a severity grade.

5. The computer-implemented method of claim 4 further comprising communicating an alert to the at least one authenticated sponsor device or the investigator client device when the severity grade meets or exceeds a prespecified threshold.

6. The computer-implemented method of claim 1 wherein transcribing the audio data further comprises analyzing at least one vocal biomarker from the audio data to derive at least one psychophysiological indicator for the participant.

7. The computer-implemented method of claim 6 further comprising generating, with the conversational AI agent, a follow-up generative voice prompt in response to the psychophysiological indicator exceeding a configurable threshold.

8. The computer-implemented method of claim 1 wherein the objective clinical data includes real-time physiological measurements received from the physiological sensor device at the participant device via a short-range wireless protocol, the real-time physiological measurements comprising at least one of heart rate, heart rate variability, physical activity level, sleep duration, or interstitial glucose.

9. The computer-implemented method of claim 1 further comprising processing, by the at least one processor, the audio data to generate an encrypted audio file comprising the audio data and an encrypted text file comprising the structured textual content.

10. The computer-implemented method of claim 1 further comprising configuring the dosage instruction according to the participant data and the protocol parameters.

11. The computer-implemented method of claim 10 wherein the dosage instruction is configured by:
(a) comparing current participant safety and efficacy markers to the one or more safety or escalation thresholds;
(b) determining an individualized titration increment that does not exceed a protocol defined maximum dose; and
(c) adjusting the individualized titration increment in response to each subsequently received set of participant data.

12. A system for managing a clinical trial of an investigational medical intervention, comprising:
at least one server comprising a processor, a non-transitory computer-readable medium, and a network interface, the non-transitory computer-readable medium comprising processor-executable instructions stored thereon that, when executed by the processor, cause the server to:
receive protocol parameters that define one or more of a dosing schema, a visit schedule, one or more safety or escalation thresholds, and participant eligibility criteria;
configure a conversational artificial intelligence (AI) model and an algorithmic logic engine in accordance with the protocol parameters,
wherein configuring the conversational AI model and the algorithmic logic engine comprises,
(i) configuring a set of generative voice prompts mapped to protocol activities for execution by a conversational AI agent, and
(ii) encoding decision rules that relate participant reported and objective clinical data to the dosing schema and the safety or escalation thresholds;
store, in an electronic record repository, the protocol parameters, the generative voice prompts, and the decision rules in a traceable format;
at least one participant device comprising a microphone, a speaker, and programmed circuitry configured to:
receive from the server a first generative voice prompt;
capture audio data from a participant in response to the first generative voice prompt; and
transmit the captured audio data to the server via a secure network connection;
an audio-processing module executable by the server and configured to:
transcribe the audio data into structured textual content; and
extract participant data comprising at least one of symptom information, adherence confirmation, or adverse event descriptors,
wherein the algorithmic logic engine is executable by the server and configured to:
evaluate the participant data in combination with objective clinical data received from at least one physiological sensor device or external laboratory feed against the safety or escalation thresholds; and
generate at least one of a dosage or scheduling instruction for the investigational medical intervention or a follow-up generative voice prompt;
a communication module executable by the server and configured to transmit the generated dosage or scheduling instruction or the follow-up generative voice prompt to the participant device;
an audit trail module executable by the server and configured to:
timestamp and immutably record in the electronic record repository (i) each generative voice prompt, (ii) each audio capture, (iii) the structured textual content, (iv) each generated dosage or scheduling instruction, and (v) metadata identifying the participant device session; and
at least one sponsor or investigator client device configured to access, via a role-based, encrypted network connection, the electronic record repository and the audit trail data for review, monitoring, or regulatory inspection.

13. The system of claim 12 wherein the dosage instruction is dynamically selected according to an adaptive dose escalation model.

14. The system of claim 13 wherein the adaptive dose escalation model comprises logic executable by the algorithmic logic engine to iteratively update a recommended next dose instruction based on accumulating participant data and objective clinical data relative to at least one protocol-defined safety and/or efficacy threshold.

15. The system of claim 12 wherein the objective clinical data includes real-time physiological measurements received from the physiological sensor device at the participant device via a short-range wireless protocol, the real-time physiological measurements comprising at least one of heart rate, heart rate variability, physical activity level, sleep duration, or interstitial glucose.

16. The system of claim 12 wherein the audio-processing module is further configured to perform sentiment analysis and vocal biomarker extraction on the received audio data to derive at least one psychophysiological indicator selected from the group consisting of stress level, fatigue, and depressive affect.

17. The system of claim 12 wherein the algorithmic logic engine is further configured to trigger a follow-up generative voice prompt when the psychophysiological indicator surpasses a configurable threshold.

18. The system of claim 12 wherein the algorithmic logic engine is further configured to enforce a cumulative dose ceiling by:
(i) maintaining, in the electronic record repository, a running total of the participant's administered dose of the investigational medical intervention over a specified interval; and
(ii) automatically suspending further upward titration and generating an electronic alert to an investigator client device when the cumulative total reaches or exceeds a predefined maximum allowable exposure.

19. The system of claim 12 wherein the algorithmic logic engine is further configured to:
(i) downgrade a dose level for the investigational medical intervention in response to detecting a dose-limiting toxicity event based on the participant data and the objective clinical data,
(ii) configure a lockout action for subsequent dose escalation, and
(iii) record the lockout action, the dose-limiting toxicity event data, and timestamp in an audit trail portion of the electronic record repository.

20. A non-transitory computer readable medium comprising processor-executable instructions stored thereon that, when executed by at least one processor, are configured to cause the at least one processor to perform one or more operations of a computer-implemented method for clinical trial management, the one or more operations comprising:
receiving protocol parameters that define one or more of a dosing schema, a visit schedule, one or more safety or escalation thresholds, and participant eligibility criteria for a clinical trial for an investigational medical intervention;
configuring a conversational artificial intelligence (AI) model in accordance with the protocol parameters,
wherein configuring the conversational AI model comprises configuring a set of generative voice prompts mapped to the protocol parameters;
configuring an algorithmic logic engine in accordance with the protocol parameters,
wherein the algorithmic logic engine comprises one or more decision rules for processing participant reported data according to the protocol parameters;
transmitting, to a participant device comprising a microphone and a speaker, a first generative voice prompt via a conversational AI agent executing the conversational AI model;
receiving, via the participant device, audio data in response to the first generative voice prompt;
transcribing the audio data into structured textual content and extracting participant data comprising at least one of symptom information, adherence confirmation, or adverse event descriptors;
evaluating, by the algorithmic logic engine, the participant data in combination with objective clinical data received from a physiological sensor device or an external laboratory data feed against the protocol parameters;
dynamically generating a second generative voice prompt in response to evaluating the participant data,
wherein the second generative voice prompt comprises a dosage instruction for the investigational medical intervention, or a follow-up generative voice prompt configured to request additional information from the participant;
recording, in an electronic record repository, (i) the protocol parameters, (ii) each generative voice prompt, (iii) the audio data, (iv) the structured textual content, (v) a timestamp, (vi) an audit trail indicator, and (vii) any dosage instruction; and
rendering the secure electronic record repository accessible to at least one authenticated sponsor device or investigator client device via a role-based network connection.
